SEQUENCE LISTING (85 sequences)

SEQ ID NO: 1 - protein, 30 aa, ARTIFICIAL SEQUENCE
  Human alphaCGRP (8-37)
VTHRLAGLLSRSGGVVKNNFVPTNVGSKAF

SEQ ID NO: 2 - protein, 31 aa, ARTIFICIAL SEQUENCE
  CGRP Peptide Analog
WVTHXLAGLLSXSGGVVXKNFVPTDVGPFAF

SEQ ID NO: 3 - protein, 31 aa, ARTIFICIAL SEQUENCE
  CGRP Peptide Analog
WVTHXLAGLLSXSGGVVXKNFVPTDVGXFAF

SEQ ID NO: 4 - protein, 31 aa, ARTIFICIAL SEQUENCE
  CGRP Peptide Analog
WVTHRLAGLLSRSGGVVKNNFVPTNVGSKAF

SEQ ID NO: 5 - protein, 31 aa, ARTIFICIAL SEQUENCE; identical to SEQ ID NO: 2
  PEGylated CGRP peptide analog

SEQ ID NO: 6 - protein, 31 aa, ARTIFICIAL SEQUENCE; identical to SEQ ID NO: 3
  PEGylated CGRP peptide analog

SEQ ID NO: 7 - protein, 31 aa, ARTIFICIAL SEQUENCE
  CGRP Peptide Analog
WVTHRLAGLLSRSGGVVRKNFVPTDVGPFAF

SEQ ID NO: 8 - protein, 31 aa, ARTIFICIAL SEQUENCE
  PEGylated CGRP peptide analog
WVTHRLAGLASRSGGVVRKNFVPTDVGPFAF

SEQ ID NO: 9 - protein, 31 aa, ARTIFICIAL SEQUENCE
  PEGylated CGRP peptide analog
WVTHRLAGLASXSGGVVRKNFVPTDVGPFAF

SEQ ID NO: 10 - protein, 31 aa, ARTIFICIAL SEQUENCE
  PEGylated CGRP peptide analog
WVTHXLAGLASXSGGVVRKNFVPTDVGPFAF

SEQ ID NO: 11 - protein, 31 aa, ARTIFICIAL SEQUENCE
  PEGylated CGRP peptide analog
WVTHRLAGLLSRPGGVVRKNFVPTDVGPFAF

SEQ ID NO: 12 - protein, 31 aa, ARTIFICIAL SEQUENCE
  PEGylated CGRP peptide analog
WVTHRLAGLASRPGGVVRKNFVPTDVGPFAF

SEQ ID NO: 13 - protein, 31 aa, ARTIFICIAL SEQUENCE
  PEGylated CGRP peptide analog
WVTHXLAGLASRPGGVVRKNFVPTDVGPFAF

SEQ ID NO: 14 - protein, 31 aa, ARTIFICIAL SEQUENCE
  PEGylated CGRP peptide analog
WVTHXLAGLLSRPGGVVRKNFVPTDVGPFAF

SEQ ID NO: 15 - protein, 31 aa, ARTIFICIAL SEQUENCE; identical to SEQ ID NO: 7
  PEGylated CGRP peptide analog

SEQ ID NO: 16 - protein, 31 aa, ARTIFICIAL SEQUENCE
  PEGylated CGRP peptide analog
WVTHXLAGLLPRSGGVVRKNFVPTDVGPFAF

SEQ ID NO: 17 - protein, 31 aa, ARTIFICIAL SEQUENCE
  PEGylated CGRP peptide analog
WVTHRLAGLLPRSGGVVRKNFVPTDVGPFAF

SEQ ID NO: 18 - protein, 31 aa, ARTIFICIAL SEQUENCE
  PEGylated CGRP peptide analog
WVTHQLAGLLSQSGGVVXKNFVPTDVGPFAF

SEQ ID NO: 19 - protein, 31 aa, ARTIFICIAL SEQUENCE
  PEGylated CGRP peptide analog
WVTHRLAGLLSRSGGVVRXNFVPTDVGPFAF

SEQ ID NO: 20 - protein, 31 aa, ARTIFICIAL SEQUENCE
  PEGylated CGRP peptide analog
WVTHRLAGLLSXSGGVVRKNFVPTDVGPFAF

SEQ ID NO: 21 - protein, 31 aa, ARTIFICIAL SEQUENCE
  PEGylated CGRP peptide analog
WVTHRXAGLLSRXGGVVRKNFVPTDVGPFAF

SEQ ID NO: 22 - protein, 31 aa, ARTIFICIAL SEQUENCE
  PEGylated CGRP peptide analog
WVEHRLKGLLSRSGGVVRKNFVPTDVGPFAF

SEQ ID NO: 23 - protein, 31 aa, ARTIFICIAL SEQUENCE
  PEGylated CGRP peptide analog
WVTHRLEGLLKRSGGVVRKNFVPTDVGPFAF

SEQ ID NO: 24 - protein, 31 aa, ARTIFICIAL SEQUENCE
  PEGylated CGRP peptide analog
XVTHXLAGLLSXSGGVVRKNFVPTDVGPFAF

SEQ ID NO: 25 - protein, 31 aa, ARTIFICIAL SEQUENCE
  PEGylated CGRP peptide analog
WVEHXLKGLLSXSGGVVRKNFVPTDVGPFAF

SEQ ID NO: 26 - protein, 31 aa, ARTIFICIAL SEQUENCE
  PEGylated CGRP peptide analog
XVEHXLKGLLSXSGGVVRKNFVPTDVGPFAF

SEQ ID NO: 27 - protein, 32 aa, ARTIFICIAL SEQUENCE
  PEGylated CGRP peptide analog
XWVEHXLKGLLSXSGGVVRKNFVPTDVGPFAF

SEQ ID NO: 28 - protein, 31 aa, ARTIFICIAL SEQUENCE; identical to SEQ ID NO: 2
  PEGylated CGRP peptide analog

SEQ ID NO: 29 - protein, 31 aa, ARTIFICIAL SEQUENCE; identical to SEQ ID NO: 2
  PEGylated CGRP peptide analog

SEQ ID NO: 30 - protein, 31 aa, ARTIFICIAL SEQUENCE; identical to SEQ ID NO: 2
  PEGylated CGRP peptide analog

SEQ ID NO: 31 - protein, 31 aa, ARTIFICIAL SEQUENCE
  CGRP peptide analog
WVTHXLAGLLSXSGGVVRKNFVPTDVGPFAF

SEQ ID NO: 32 - protein, 31 aa, ARTIFICIAL SEQUENCE; identical to SEQ ID NO: 31
  PEGylated CGRP peptide analog

SEQ ID NO: 33 - protein, 31 aa, ARTIFICIAL SEQUENCE; identical to SEQ ID NO: 2
  PEGylated CGRP peptide analog

SEQ ID NO: 34 - protein, 32 aa, ARTIFICIAL SEQUENCE
  PEGylated CGRP peptide analog
WVTHQLAGLLSXSGGVVRXKNFVPTDVGPFAF

SEQ ID NO: 35 - protein, 32 aa, ARTIFICIAL SEQUENCE
  PEGylated CGRP peptide analog
WVTHXLAGLLSQSGGVVRXKNFVPTDVGPFAF

SEQ ID NO: 36 - protein, 32 aa, ARTIFICIAL SEQUENCE
  PEGylated CGRP peptide analog
WVTHXLAGLLSXSGGVVRXKNFVPTDVGPFAF

SEQ ID NO: 37 - protein, 32 aa, ARTIFICIAL SEQUENCE; identical to SEQ ID NO: 36
  PEGylated CGRP peptide analog

SEQ ID NO: 38 - protein, 32 aa, ARTIFICIAL SEQUENCE
  PEGylated CGRP peptide analog
WVEHRLKGLLSXSGGVVRXKNFVPTDVGPFAF

SEQ ID NO: 39 - protein, 32 aa, ARTIFICIAL SEQUENCE
  PEGylated CGRP peptide analog
WVEHXLKGLLSXSGGVVRXKNFVPTDVGPFAF

SEQ ID NO: 40 - protein, 31 aa, ARTIFICIAL SEQUENCE
  PEGylated CGRP peptide analog
WVEHXLKGLLSXSGGVVXKNFVPTDVGPFAF

SEQ ID NO: 41 - protein, 32 aa, ARTIFICIAL SEQUENCE; identical to SEQ ID NO: 38
  PEGylated CGRP peptide analog

SEQ ID NO: 42 - protein, 32 aa, ARTIFICIAL SEQUENCE
  PEGylated CGRP peptide analog
WVEHRLKGLLSQSGGVVRXKNFVPTDVGPFAF

SEQ ID NO: 43 - protein, 37 aa, Homo sapiens
  MOD_RES (37)..(37): AMIDATION
ACDTATCVTHRLAGLLSRSGGVVKNNFVPTNVGSKAF

SEQ ID NO: 44 - protein, 31 aa, ARTIFICIAL SEQUENCE
  PEGylated CGRP peptide analog
WVTHXLAGLLSXSGGVVXKNFVPTDVGPXAF

SEQ ID NO: 45 - protein, 31 aa, ARTIFICIAL SEQUENCE; identical to SEQ ID NO: 2
  PEGylated CGRP peptide analog

SEQ ID NO: 46 - protein, 31 aa, ARTIFICIAL SEQUENCE; identical to SEQ ID NO: 44
  PEGylated CGRP peptide analog

SEQ ID NO: 47 - protein, 31 aa, ARTIFICIAL SEQUENCE; identical to SEQ ID NO: 44
  PEGylated CGRP peptide analog

SEQ ID NO: 48 - protein, 31 aa, ARTIFICIAL SEQUENCE; identical to SEQ ID NO: 44
  PEGylated CGRP peptide analog

SEQ ID NO: 49 - protein, 31 aa, ARTIFICIAL SEQUENCE
  PEGylated CGRP peptide analog
WVTHXLAGLLSXSGGVVXKNXVPTDVGPXAF

SEQ ID NO: 50 - protein, 6 aa, ARTIFICIAL SEQUENCE
  Linker sequence
XXNXXG

SEQ ID NO: 51 - protein, 8 aa, ARTIFICIAL SEQUENCE
  Peptidyl linker sequence
GGGGGGGG

SEQ ID NO: 52 - protein, 5 aa, ARTIFICIAL SEQUENCE
  Peptidyl linker sequence
GGGGG

SEQ ID NO: 53 - protein, 7 aa, ARTIFICIAL SEQUENCE
  Peptidyl linker sequence
GGGGGGG

SEQ ID NO: 54 - protein, 5 aa, ARTIFICIAL SEQUENCE
  Peptidyl linker sequence
GGGGS

SEQ ID NO: 55 - protein, 6 aa, ARTIFICIAL SEQUENCE
  Peptidyl linker sequence
GGGGGK

SEQ ID NO: 56 - protein, 7 aa, ARTIFICIAL SEQUENCE
  Peptidyl linker sequence
GGGGGKR

SEQ ID NO: 57 - protein, 8 aa, ARTIFICIAL SEQUENCE
  Peptidyl linker sequence
GGGKGGGG

SEQ ID NO: 58 - protein, 8 aa, ARTIFICIAL SEQUENCE
  Peptidyl linker sequence
GGGNGSGG

SEQ ID NO: 59 - protein, 8 aa, ARTIFICIAL SEQUENCE
  Peptidyl linker sequence
GGGCGGGG

SEQ ID NO: 60 - protein, 5 aa, ARTIFICIAL SEQUENCE
  Peptidyl linker sequence
GPNGG

SEQ ID NO: 61 - protein, 8 aa, ARTIFICIAL SEQUENCE
  Peptidyl linker sequence
GGGLGGGG

SEQ ID NO: 62 - protein, 5 aa, ARTIFICIAL SEQUENCE
  Linker sequence
GGGGS

SEQ ID NO: 63 - protein, 10 aa, ARTIFICIAL SEQUENCE
  Linker sequence
GGGGSGGGGS

SEQ ID NO: 64 - protein, 25 aa, ARTIFICIAL SEQUENCE
  Linker sequence
GGGGSGGGGSGGGGSGGGGSGGGGS

SEQ ID NO: 65 - protein, 6 aa, ARTIFICIAL SEQUENCE
  Peptide linker sequence
GGEGGG

SEQ ID NO: 66 - protein, 8 aa, ARTIFICIAL SEQUENCE
  Peptide linker sequence
GGEEEGGG

SEQ ID NO: 67 - protein, 5 aa, ARTIFICIAL SEQUENCE
  Peptide linker sequence
GEEEG

SEQ ID NO: 68 - protein, 4 aa, ARTIFICIAL SEQUENCE
  Peptide linker sequence
GEEE

SEQ ID NO: 69 - protein, 6 aa, ARTIFICIAL SEQUENCE
  Peptide linker sequence
GGDGGG

SEQ ID NO: 70 - protein, 7 aa, ARTIFICIAL SEQUENCE
  Peptide linker sequence
GGDDDGG

SEQ ID NO: 71 - protein, 5 aa, ARTIFICIAL SEQUENCE
  Peptide linker sequence
GDDDG

SEQ ID NO: 72 - protein, 4 aa, ARTIFICIAL SEQUENCE
  Peptide linker sequence
GDDD

SEQ ID NO: 73 - protein, 21 aa, ARTIFICIAL SEQUENCE
  Peptide linker sequence
GGGGSDDSDEGSDGEDGGGGS

SEQ ID NO: 74 - protein, 5 aa, ARTIFICIAL SEQUENCE
  Peptide linker sequence
WEWEW

SEQ ID NO: 75 - protein, 5 aa, ARTIFICIAL SEQUENCE
  Peptide linker sequence
FEFEF

SEQ ID NO: 76 - protein, 6 aa, ARTIFICIAL SEQUENCE
  Peptide linker sequence
EEEWWW

SEQ ID NO: 77 - protein, 6 aa, ARTIFICIAL SEQUENCE
  Peptide linker sequence
EEEFFF

SEQ ID NO: 78 - protein, 7 aa, ARTIFICIAL SEQUENCE
  Peptide linker sequence
WWEEEWW

SEQ ID NO: 79 - protein, 7 aa, ARTIFICIAL SEQUENCE
  Peptide linker sequence
FFEEEFF

SEQ ID NO: 80 - protein, 6 aa, ARTIFICIAL SEQUENCE
  Peptide linker sequence
XXYXXG

SEQ ID NO: 81 - protein, 6 aa, ARTIFICIAL SEQUENCE
  Peptide linker sequence
XXSXXG

SEQ ID NO: 82 - protein, 6 aa, ARTIFICIAL SEQUENCE
  Peptide linker sequence
XXTXXG

SEQ ID NO: 83 - protein, 22 aa, ARTIFICIAL SEQUENCE
  Peptidyl linker sequence
GSGSATGGSGSTASSGSGSATH

SEQ ID NO: 84 - protein, 22 aa, ARTIFICIAL SEQUENCE
  Peptidyl linker sequence
HGSGSATGGSGSTASSGSGSAT

SEQ ID NO: 85 - protein, 19 aa, ARTIFICIAL SEQUENCE
  Rigid peptidyl linker sequence
AEAAAKEAAAKEAAAKAGG

(19) United States
(12) Patent Application Publication
(10) Pub. No.: US 2009/0252703 A1
(43) Pub. Date: Oct. 8, 2009

(54) Use of alcohol co-solvents to improve pegylation reaction yields

(76) Inventors: Colin V. Gegg, JR., Newbury Park, CA (US); Derin C. D'Amico, Newbury Park, CA (US); Marie E. Wright, Westlake Village, CA (US)

Correspondence Address: AMGEN INC., MAIL STOP 28-2-C, ONE AMGEN CENTER DRIVE, THOUSAND OAKS, CA, 91320-1799, US

(21) Appl. No.: 11/974,472

(22) Filed: Oct. 12, 2007

Related U.S. Application Data
(60) Provisional application No. 60/853,132, filed on Oct. 19, 2006.

(51) Int. Cl. A61K 38/17; A61K 38/28; A61K 38/20

(52) U.S. Cl. 424/85.2; 514/12; 514/3

(57) ABSTRACT

Disclosed is a method of producing a composition of matter. The method involves obtaining a pharmacologically active peptide; and conjugating the peptide to a pharmaceutically acceptable polyethylene glycol (PEG) by reacting the peptide with a PEG-aldehyde compound at a free amine moiety on the peptide in a buffer solution comprising an alcohol co-solvent.

This application claims priority from U.S. Provisional Application No. 60/853,132, filed Oct. 19, 2006, which is hereby incorporated by reference.

This application is related to U.S. Non-provisional application Ser. No. 11/584,177, filed Oct. 19, 2006, which claims priority from U.S. Provisional Application No. 60/729,083, filed Oct. 21, 2005, both of which applications are hereby incorporated by reference.

This application incorporates by reference all subject matter on the diskette containing a computer readable form (CFR) sequence listing, which is identified by the file name, A-1193-US-NP.ST25.txt, created on Oct. 12, 2007, the size of which file is 43 KB.

Throughout this application various publications are referenced within parentheses. The disclosures of these publications in their entireties are hereby incorporated by reference in this application in order to more fully describe the state of the art to which this invention pertains.

### BACKGROUND OF THE INVENTION

1. Field of the Invention

The present invention is related to the biochemical arts, in particular to PEGylation of therapeutic peptides.

2. Discussion of the Related Art

In general, therapeutic peptides and proteins exhibit very fast plasma clearance, thus requiring frequent injections to ensure steady pharmaceutically relevant blood levels of a particular peptide or protein with pharmacological activity. Many pharmaceutically relevant peptides and proteins, even those having human primary structure, can be immunogenic, giving rise to neutralizing antibodies circulating in the bloodstream. This is especially true for intravenous and subcutaneous administration, which is of particular concern for the delivery of most peptide and protein drugs.

By increasing the molecular volume and by masking potential epitopes, modification of a therapeutic polypeptide with a vehicle, such as a polyethylene glycol (PEG) polymer, has been shown to be effective in reducing both the rate of clearance as well as the antigenicity of the protein. Reduced proteolysis, increased water solubility, reduced renal clearance, and steric hindrance to receptor-mediated clearance are a number of mechanisms by which the attachment of a polymer to the backbone of a polypeptide may prove beneficial in enhancing the pharmacokinetic properties of the drug. For example, Davis et al. taught conjugating PEG or polypropylene glycol to proteins such as enzymes and insulin to produce a less immunogenic product while retaining a substantial proportion of the biological activity. (U.S. Pat. No. 4,179,337).

Covalent conjugation of therapeutic proteins with poly(ethylene glycol) (PEG) has been widely recognized as an approach to significantly extend the in vivo circulating half-lives of therapeutic proteins. (E.g., Caliceti et al., Pharmacokinetic and biodistribution properties of poly(ethyleneglycol)-protein conjugates, Advanced Drug Delivery Reviews 55:1261-77 (2003); Greenwald, PEG drugs: an overview, Journal of Controlled Release 74:159-71 (2001)). PEGylation achieves this effect predominately by retarding renal clearance, since the PEG moiety adds considerable hydrodynamic radius to the protein. (Zalipsky, S., et al., Use of functionalized poly(ethylene glycol)s for modification of polypeptides, in poly(ethylene glycol) chemistry: Biotechnical and biomedical applications, J. M. Harris, Ed., Plenum Press: New York., 347-370 (1992)). Additional benefits often conferred by PEGylation of proteins include increased solubility, resistance to proteolytic degradation, and reduced immunogenicity of the therapeutic polypeptide. (E.g., J. M. Harris et al., PEGylation: A Novel Process for Modifying Pharmacokinetics, Clin. Pharmacokinet., 40:539-551 (2001); and R. Mehvar, Modulation of the Pharmacokinetics and Pharmacodynamics of Proteins by Polyethylene Glycol Conjugation, J. Pharm. Pharmaceut. Sci., 3:125-136 (2000); Harris and Chess, Effect of PEGylation on pharmaceuticals, Nature Reviews Drug Discovery 2:214-20 (2003)). The merits of protein PEGylation are evidenced by the commercialization of several PEGylated proteins including PEG-Adenosine deaminase (Adagen™/Enzon Corp.), PEG-L-asparaginase (Oncaspar™/Enzon Corp.), PEG-Interferon α-2b (PEG-Intron™/Schering/Enzon), PEG-Interferon α-2a (PEGASYS™/Roche) and PEG-G-CSF (Neulasta™/Amgen) as well as many others in clinical trials. (See, e.g., Sannes, L J, Using PEG Technology for strategic advantage, Spectrum Therapy Markets and Emerging Technologies, May 14, 2004).

The use of trifluoroethanol (TFE) to affect secondary and tertiary structure of polypeptides has been described in the scientific literature. For example, the Alzheimer's protein Aβ1-40 is predominantly in the monomeric form in TFE, as determined by NMR. (Filippov, A.; Sulejmanova, A.; Antzutkin, O.; Groebner, G., Diffusion and aggregation of Alzheimer's Abl-40 peptide in aqueous trifluoroethanol solutions as studied by pulsed field gradient NMR. Applied Magnetic Resonance 29(3): 439-449 (2005)). As the water content is increased, the peptide begins to self-associate into higher oligomers. In another example, the Proadrenomedullin N-Terminal 20 Peptide was shown to have two different conformations in solution depending on solvent effects. (Lucyk, S.; Taha, H.; Yamamoto, H.; Miskolzie, M.; Kotovych, G., NMR conformational analysis of proadrenomedullin N-terminal 20 peptide, a proangiogenic factor involved in tumor growth. Biopolymers 81(4): 295-308 (2006)). In TFE-water, this peptide forms stable α-helix from Arg2 to Arg20, whereas in detergent-containing water, a stable helix was observed from Arg2 to Arg17. The impact of TFE on secondary peptide structure was further documented in the work of Zhao. (Zhao, J.-H.; Liu, H.-L., The effects of various alcohols on the secondary structural integrity of melittin, TH-10Aox, and Tc1 by molecular dynamics simulations. Chemical Physics Letters 420(1-3): 235-240 (2006)). A related application of TFE is its use in HPLC mobile phases in the study of protein and peptides; TFE increases the efficiency of purification and identification by decreasing the amount of oligomer formation. (Bidlingmeyer, B.; Wang, Q. Additives for reversed-phase HPLC mobile phases, U.S. 2005/0011836 A1).

Examples of the reductive amination of small molecule amines and aldehydes in the presence of TFE are scarce, but known. (Nesi, M.; Borghi, D.; Brasca, M. G.; Fiorentini, F.; Pevarello, P., A practical synthesis of the major 3-hydroxy-2-pyrrolidinone metabolite of a potent CDK2/cyclin A inhibitor. Bioorganic & Medicinal Chemistry Letters 16(12): 3205-3208 (2006); Bunlaksananusorn, T.; Rampf, F., A facile one-pot synthesis of chiral b-amino esters. Synlett (17): 2682-2684 (2005)).

It is a desideratum to combine the therapeutic benefits of PEG-conjugated therapeutic peptides and analogs, such as substantially increased pharmacological half-life and decreased immunogenicity, with an increased efficiency of reductive amination that makes production of PEGylated peptides on a large scale more practical and commercially feasible. These and other benefits are provided by the present invention.

### SUMMARY OF THE INVENTION

The present invention relates to a method of producing a composition of matter involving a pharmacologically active peptide. The method includes the steps of: (a) obtaining a pharmacologically active peptide; and (b) conjugating the obtained pharmacologically active peptide to a pharmaceutically acceptable polyethylene glycol (PEG) by reacting the peptide with a PEG-aldehyde compound at a free amine moiety (e.g., a primary or secondary amine) on the peptide, in a buffer solution comprising an alcohol co-solvent, whereby the composition of matter is produced.

The use of the inventive method is particularly beneficial for PEGylating peptides that are relatively insoluble in an aqueous medium, typically peptides with aqueous solubility below about 0.1 to 10 mg/mL, or in some cases, below about 1 to 10 mg/mL.

Additional benefits of the inventive method include accelerating the PEGylation reaction and improving PEGylation efficiency for an otherwise recalcitrant peptide by changing its conformation in solution into a form in which the reactive amine is more accessible to the PEGylating reagent.

By employing the method of the invention a significant benefit can be realized in the efficiency of peptide PEGylation by reductive amination, which can be translated into lower production costs for therapeutic peptide-based medicines intended for the treatment of patients with grievous illness.

### BRIEF DESCRIPTION OF THE FIGURES

FIG. 1 illustrates the effects of some different alcohol co-solvents on reaction yields for PEGylation of a relatively soluble CGRP peptide (SEQ. ID NO:7).

FIG. 2 shows a comparison of the effects of IPA and TFE on the PEGylation efficiency of a relatively soluble CGRP peptide Ac-WVTHRLAGLLSRSGGVVRKNFVPTDVGPFAF-NH₂ (SEQ ID NO:7) and a relatively insoluble CGRP peptide (SEQ ID NO:31).

FIG. 3 shows the effect of co-solvent on the PEGylation efficiency of a relatively soluble CGRP peptide (SEQ. ID NO:7) in the presence of IPA [symbol] or TFE [symbol] or, HFIPA

[symbol]

FIG. 4 shows the effect of co-solvent concentration on the PEGylation efficiency of a relatively insoluble CGRP peptide (SEQ ID NO:2) in the presence of IPA [symbol] , or TFE [symbol] , or HFIPA [symbol] .

FIG. 5 shows the effect of co-solvent concentration on the PEGylation efficiency of a relatively insoluble CGRP peptide (SEQ ID NO:3) in the presence of IPA [symbol] or TFE [symbol] , or HFIPA [symbol] .

### DETAILED DESCRIPTION OF THE PREFERRED EMBODIMENTS

The present invention relates to a method of producing a composition of matter involving a pharmacologically active peptide. In accordance with the inventive method, the types of peptides and proteins that would undergo more efficient PEGylation by reductive amination due to the inclusion of an alcohol co-solvent to the reaction buffer solution are hydrophobic and hydrophilic in nature, with a MW range from about 1 kD to about 100 kD, or greater.

The terms used throughout this specification are defined as follows, unless otherwise limited in specific instances. As used in the specification and the appended claims, the singular forms “a”, “an”, and “the” include plural referents unless the context clearly dictates otherwise.

“Peptide”, “polypeptide” and “protein” are used interchangeably herein and include a molecular chain of amino acids linked through peptide bonds. The terms do not refer to a specific length of the product. Thus, “peptides,” and “oligopeptides,” are included within the definition of polypeptide. The terms include post-translational modifications of the peptide, for example, glycosylations, acetylations, phosphorylations and the like. In addition, protein fragments, analogs, mutated or variant proteins, fusion proteins, for example fusions of a pharmacologically active peptide with an Fc domain of an immunoglobulin, antibodies, antibody peptides and the like are included within the meaning of polypeptide. The terms also include molecules in which one or more amino acid analogs or non-canonical or unnatural amino acids are included as can be synthesized, or expressed recombinantly using known protein engineering techniques. In addition, inventive fusion proteins can be derivatized as described herein by well-known organic chemistry techniques.

The term “recombinant” indicates that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well known molecular biological procedures. A “recombinant DNA molecule,” is comprised of segments of DNA joined together by means of such molecular biological techniques. The term “recombinant protein” or “recombinant polypeptide” as used herein refers to a protein molecule which is expressed using a recombinant DNA molecule. A “recombinant host cell” is a cell that contains and/or expresses a recombinant nucleic acid.

A “polynucleotide sequence” or “nucleotide sequence” or “nucleic acid sequence,” as used interchangeably herein, is a polymer of nucleotides, including an oligonucleotide, a DNA, and RNA, a nucleic acid, or a character string representing a nucleotide polymer, depending on context. From any specified polynucleotide sequence, either the given nucleic acid or the complementary polynucleotide sequence can be determined. Included are DNA or RNA of genomic or synthetic origin which may be single- or double-stranded, and represent the sense or antisense strand.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of ribonucleotides along the mRNA chain, and also determines the order of amino acids along the polypeptide (protein) chain. The DNA sequence thus codes for the RNA sequence and for the amino acid sequence.

“Expression of a gene” or “expression of a nucleic acid” means transcription of DNA into RNA (optionally including modification of the RNA, e.g., splicing), translation of RNA into a polypeptide (possibly including subsequent post-translational modification of the polypeptide), or both transcription and translation, as indicated by the context.

The term “gene” is used broadly to refer to any nucleic acid associated with a biological function. Genes typically include coding sequences and/or the regulatory sequences required for expression of such coding sequences. The term “gene” applies to a specific genomic or recombinant sequence, as well as to a cDNA or mRNA encoded by that sequence. A “fusion gene” contains a coding region that encodes a fusion protein. Genes also include non-expressed nucleic acid segments that, for example, form recognition sequences for other proteins. Non-expressed regulatory sequences including transcriptional control elements to which regulatory proteins, such as transcription factors, bind, resulting in transcription of adjacent or nearby sequences.

As used herein the term “coding region” when used in reference to a structural gene refers to the nucleotide sequences which encode the amino acids found in the nascent polypeptide as a result of translation of an mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

Transcriptional control signals in eukaryotes comprise “promoter” and “enhancer” elements. Promoters and enhancers consist of short arrays of DNA sequences that interact specifically with cellular proteins involved in transcription (Maniatis, et al., Science 236:1237 (1987)). Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see Voss, et al., Trends Biochem. Sci., 11:287 (1986) and Maniatis, et al., Science 236:1237 (1987)).

The term “expression vector” as used herein refers to a recombinant DNA molecule containing a desired coding sequence and appropriate nucleic acid sequences necessary for the expression of the operably linked coding sequence in a particular host cell. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence for the inventive recombinant fusion protein, so that the expressed fusion protein can be secreted by the recombinant host cell, for more facile isolation of the fusion protein from the cell, if desired. Such techniques are well known in the art. (E.g., Goodey, Andrew R.; et al., Peptide and DNA sequences, U.S. Pat. No. 5,302,697; Weiner et al., Compositions and methods for protein secretion, U.S. Pat. No. 6,022,952 and U.S. Pat. No. 6,335,178; Uemura et al., Protein expression vector and utilization thereof, U.S. Pat. No. 7,029,909; Ruben et al., 27 human secreted proteins, US 2003/0104400 A1).

The terms “in operable combination”, “in operable order” and “operably linked” as used herein refer to the linkage of nucleic acid sequences in such a manner or orientation that a nucleic acid molecule capable of directing the transcription of a given gene and/or the synthesis of a desired protein molecule is produced. The term also refers to the linkage of amino acid sequences in such a manner so that a functional protein is produced and/or transported.

The term “fusion protein” indicates that the protein includes polypeptide components derived from more than one parental protein or polypeptide. Typically, a fusion protein is expressed from a fusion gene in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell as a single protein.

A “domain” of a protein is any portion of the entire protein, up to and including the complete protein, but typically comprising less than the complete protein. A domain can, but need not, fold independently of the rest of the protein chain and/or be correlated with a particular biological, biochemical, or structural function or location (e.g., a ligand binding domain, or a cytosolic, transmembrane or extracellular domain).

“Antibody” or “antibody peptide(s)” refer to an intact antibody, or a binding fragment thereof that competes with the intact antibody for specific binding and includes chimeric, humanized, fully human, and bispecific antibodies. In certain embodiments, binding fragments are produced by recombinant DNA techniques. In additional embodiments, binding fragments are produced by enzymatic or chemical cleavage of intact antibodies. Binding fragments include, but are not limited to, Fab, Fab′, F(ab′)₂, Fv, and single-chain antibodies.

The term “Fc domain” encompasses native Fc and Fc variant molecules. As with Fc variants and native Fc's, the term “Fc domain” includes molecules in monomeric or multimeric form, whether digested from whole antibody or produced by other means. In one embodiment, the Fc domain is a human native Fc domain. In other embodiments, the “Fc domain” can be a Fc variant, an analog, a mutant, a truncation, or a derivative of human Fc or of an alternative mammalian Fc polypeptide.

The term “native Fc” refers to a molecule or sequence comprising the amino acid sequence of a non-antigen-binding fragment resulting from digestion of whole antibody, whether in monomeric or multimeric form, at which a peptide may be added or conjugated by being covalently bound, directly or indirectly through a linker, to a loop region of the Fc domain. The original immunoglobulin source of the native Fc is preferably of human origin (although non-human mammalian native Fc is included in “native Fc” and can also be useful in some embodiments), and may be any of the immunoglobulins, although IgG1 and IgG2 are preferred. The native Fc may optionally comprise an amino terminal methionine residue. Native Fcs are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms. A “monovalent dimeric” Fc-peptide fusion, or “monovalent dimer”, is a Fc-peptide fusion that includes a therapeutic peptide conjugated with only one of the dimerized Fc domains. A “bivalent dimeric” Fc-peptide fusion, or “bivalent dimer”, is a Fc-peptide fusion having both of the dimerized Fc domains each conjugated separately with a therapeutic peptide (the same or different therapeutic peptides).

The term “Fc variant” refers to a molecule or sequence that is modified from a native Fc but still comprises a binding site for the salvage receptor, FcRn. International applications WO 97/34631 (published 25 Sep. 1997) and WO 96/32478 describe exemplary Fc variants, as well as interaction with the salvage receptor, and are hereby incorporated by reference. Thus, the term “Fc variant” comprises a molecule or sequence that is humanized from a non-human native Fc. Furthermore, a native Fc comprises sites that may be removed because they provide structural features or biological activity that are not required for molecules of the present invention. Thus, the term “Fc variant” comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

The term “pharmacologically active” means that a substance so described is determined to have activity that affects a medical parameter (e.g., blood pressure, blood cell count, cholesterol level) or disease state (e.g., cancer, autoimmune disorders, neurological disorders, chronic pain). Thus, pharmacologically active peptides or polypeptides include agonistic or mimetic and antagonistic peptides, including antibodies, antibody peptides, fusions with an Fc domain. Pharmacologically active peptides include polypeptide molecules of greater than about 10 amino acid residues, whether existing in nature or not, provided that such molecules are not membrane-bound. Exemplary pharmacologically active peptides include insulin, interleukin (IL)-1ra, leptin, soluble tumor necrosis factor (TNF) receptors type 1 and type 2 (sTNF-R1, sTNF-R2), keratinocyte growth factor (KGF), erythropoietin (EPO), thrombopoietin (TPO) and TPO-mimetic peptides, granulocyte colony-stimulating factor (G-CSF), darbepoietin, glial cell line-derived neurotrophic factor (GDNF), calcitonin (CT), amylin (AMY), adrenomedullin (ADM), and calcitonin gene-related peptide (CGRP) peptide antagonists, BAFF antagonist peptides, ang-2-binding peptides, NGF-binding peptides, myostatin-binding peptides, toxin peptides, and the like.

The terms “-mimetic peptide” and “-agonist peptide” refer, respectively, to a peptide or polypeptide having biological activity comparable to a protein (e.g., EPO, TPO, G-CSF) of interest or to a peptide or polypeptide that interacts as an agonist with a particular protein of interest. These terms further include peptides or polypeptides that indirectly mimic the activity of a protein of interest, such as by potentiating the effects of the natural ligand of the protein of interest; see, for example, the EPO-mimetic peptides listed in U.S. Pat. No. 6,660,843, which is hereby incorporated by reference. Thus, the term “EPO-mimetic peptide” comprises any peptides or polypeptides that can be identified or derived as described in Wrighton et al. (1996), Science 273: 458-63, Naranda et al. (1999), Proc. Natl. Acad. Sci. USA 96: 7569-74, or any other EPO-mimetic peptide sequences identified in Table 5 of U.S. Ser. No. 11/502,761, titled Modified Fc Molecules, filed Aug. 10, 2006, by Gegg et al., which claims the benefit of U.S. Provisional Application No. 60/707,842, filed Aug. 12, 2005, both of which are incorporated herein by reference in their entireties. Those of ordinary skill in the art appreciate that each of these references enables one to select different peptides or polypeptides than actually disclosed therein by following the disclosed procedures with different peptide libraries.

The term “-antagonist peptide” or “inhibitor peptide” refers to a peptide that blocks or in some way interferes with the biological activity of the associated protein of interest, or has biological activity comparable to a known antagonist or inhibitor of the associated protein of interest.

The term “BAFF-antagonist peptide” comprises peptides that can be identified or derived as described in U.S. Pat. Appln. No. 2003/0195156 A1, which is incorporated herein by reference. Those of ordinary skill in the art appreciate that the foregoing reference enables one to select different peptides than actually disclosed therein by following the disclosed procedures with different peptide libraries. Other useful BAFF-antagonist peptides appear in Table 10 of U.S. Ser. No. 11/502,761, titled Modified Fc Molecules, filed Aug. 10, 2006, by Gegg et al., which claims the benefit of U.S. Provisional Application No. 60/707,842, filed Aug. 12, 2005, both of which are incorporated herein by reference in their entireties.

The term “peptide analog” refers to a peptide having a sequence that differs from a peptide sequence existing in nature by at least one amino acid residue substitution, internal addition, or internal deletion of at least one amino acid, and/or amino- or carboxy-terminal end truncations, or additions). An “internal deletion” refers to absence of an amino acid from a sequence existing in nature at a position other than the N- or C-terminus. Likewise, an “internal addition” refers to presence of an amino acid in a sequence existing in nature at a position other than the N- or C-terminus.

“Toxin peptides” include peptides and polypeptides having the same amino acid sequence of a naturally occurring pharmacologically active peptide or polypeptide that can be isolated from a venom, and also include modified peptide analogs of such naturally occurring molecules. (See, e.g., Kalman et al., ShK-Dap22, a potent Kv1.3-specific immunosuppressive polypeptide, J. Biol. Chem. 273(49):32697-707 (1998); Kem et al., U.S. Pat. No. 6,077,680; Mouhat et al., OsK1 derivatives, WO 2006/002850 A2; Chandy et al., Analogs of SHK toxin and their uses in selective inhibition of Kv1.3 potassium channels, WO 2006/042151; Sullivan et al., Toxin peptide therapeutic agents, WO 2006/116156 A2 and US 2007/0071764, each of which publications is incorporated herein by reference). Snakes, scorpions, spiders, bees, snails and sea anemone are a few examples of organisms that produce venom that can serve as a rich source of small bioactive toxin peptides or “toxins” that potently and selectively target ion channels and receptors. “Toxin peptide analogs”, such as, but not limited to, an OSK1 peptide analog, ShK peptide analog, or ChTx peptide analog, contain modifications of a native toxin peptide sequence of interest (e.g., amino acid residue substitutions, internal additions or insertions, internal deletions, and/or amino- or carboxy-terminal end truncations, or additions as previously described above) relative to a native toxin peptide sequence of interest.

The toxin peptides are usually between about 20 and about 80 amino acids in length, contain 2-5 disulfide linkages and form a very compact structure. Toxin peptides (e.g., from the venom of scorpions, sea anemones and cone snails) have been isolated and characterized for their impact on ion channels. Such peptides appear to have evolved from a relatively small number of structural frameworks that are particularly well suited to addressing the critical issues of potency and stability. The majority of scorpion and Conus toxin peptides, for example, contain 10-40 amino acids and up to five disulfide bonds, forming extremely compact and constrained structure (microproteins) often resistant to proteolysis. The conotoxin and scorpion toxin peptides can be divided into a number of superfamilies based on their disulfide connections and peptide folds. The solution structure of many of these has been determined by NMR spectroscopy, illustrating their compact structure and verifying conservation of their family fold. (E.g., Tudor et al., Ionisation behaviour and solution properties of the potassium-channel blocker ShK toxin, Eur. J. Biochem. 251(1-2):133-41 (1998); Pennington et al., Role of disulfide bonds in the structure and potassium channel blocking activity of ShK toxin, Biochem. 38(44): 14549-58 (1999); Jaravine et al., Three-dimensional structure of toxin OSK1 from Orthochirus scrobiculosus scorpion venom, Biochem. 36(6):1223-32 (1997); del Rio-Portillo et al.; NMR solution structure of Cn12, a novel peptide from the Mexican scorpion Centruroides noxius with a typical beta-toxin sequence but with alpha-like physiological activity, Eur. J. Biochem. 271(12): 2504-16 (2004); Prochnicka-Chalufour et al., Solution structure of discrepin, a new K+-channel blocking peptide from the alpha-KTx15 subfamily, Biochem. 45(6):1795-1804 (2006)). Examples of pharmacologically active toxin peptides for which the practice of the present invention can be useful include, but are not limited to ShK, OSK1, charybdotoxin (ChTx), kaliotoxin1 KTX1), or maurotoxin, or toxin peptide analogs of any of these, modified from the native sequences at one or more amino acid residues. Other examples are known in the art, or can be found in WO 2006/116156 A2, published Nov. 2, 2006, and U.S. patent application Ser. No. 11/406,454 (filed on Apr. 17, 2006, published Mar. 29, 2007 as US 2007/0071764, Toxin Peptide Therapeutic Agents), by Sullivan et al., both of which are incorporated by reference in their entireties, or in U.S. Provisional Application No. 60/854,674, filed Oct. 25, 2006, and U.S. Provisional Application No. 60/995,370, filed Sep. 25, 2007, both of which are incorporated by reference in their entireties.

The term “TPO-mimetic peptide” comprises peptides that can be identified or derived as described in Cwirla et al. (1997), Science 276: 1696-9, U.S. Pat. Nos. 5,869,451 and 5,932,946, which are incorporated by reference; U.S. Pat. App. No. 2003/0176352, published Sep. 18, 2003, which is incorporated by reference; WO 03/031589, published Apr. 17, 2003; WO 00/24770, published May 4, 2000; and any peptides appearing in Table 6 of U.S. Ser. No. 11/502,761, titled Modified Fc Molecules, filed Aug. 10, 2006, by Gegg et al., which claims the benefit of U.S. Provisional Application No. 60/707,842, filed Aug. 12, 2005, both of which are incorporated herein by reference in their entireties. Those of ordinary skill in the art appreciate that each of these references enables one to select different peptides than actually disclosed therein by following the disclosed procedures with different peptide libraries.

The term “ang-2-binding peptide” comprises peptides that can be identified or derived as described in U.S. Pat. App. No. 2003/0229023, published Dec. 11, 2003; WO 03/057134, published Jul. 17, 2003; U.S. 2003/0236193, published Dec. 25, 2003 (each of which is incorporated herein by reference); and any peptides appearing in Table 7 of U.S. Ser. No. 11/502,761, titled Modified Fc Molecules, filed Aug. 10, 2006, by Gegg et al., which claims the benefit of U.S. Provisional Application No. 60/707,842, filed Aug. 12, 2005, both of which are incorporated herein by reference in their entireties. Those of ordinary skill in the art appreciate that each of these references enables one to select different peptides than actually disclosed therein by following the disclosed procedures with different peptide libraries.

The term “NGF-binding peptide” comprises peptides that can be identified or derived as described in WO 04/026329, published Apr. 1, 2004 and any peptides identified in Table 8 of U.S. Ser. No. 11/502,761, titled Modified Fc Molecules, filed Aug. 10, 2006, by Gegg et al., which claims the benefit of U.S. Provisional Application No. 60/707,842, filed Aug. 12, 2005, both of which are incorporated herein by reference in their entireties. Those of ordinary skill in the art appreciate that this reference enables one to select different peptides than actually disclosed therein by following the disclosed procedures with different peptide libraries. The term “myostatin-binding peptide” comprises peptides that can be identified or derived as described in U.S. Ser. No. 10/742,379, filed Dec. 19, 2003, which is incorporated herein by reference, and peptides appearing in Table 9 of U.S. Ser. No. 11/502,761, titled Modified Fc Molecules, filed Aug. 10, 2006, by Gegg et al., which claims the benefit of U.S. Provisional Application No. 60/707,842, filed Aug. 12, 2005, both of which are incorporated herein by reference in their entireties. Those of ordinary skill in the art appreciate that each of these references enables one to select different peptides than actually disclosed therein by following the disclosed procedures with different peptide libraries.

A “CGRP peptide” is a peptide that preferentially binds the CGRP₁ receptor under physiological conditions of temperature, pH, and ionic strength. For purposes of the present invention, CGRP peptides include those having a full native CGRP peptide sequence and non-native CGRP peptide analogs containing modifications of a native CGRP sequence (e.g., amino acid substitutions, insertions, deletions, and/or amino terminal end truncations as further described herein below) relative to a native CGRP sequence of interest, which can be, e.g., any known mammalian CGRP sequence, such as but not limited to, the native human αCGRP sequence or human βCGRP sequence. In accordance with the present invention, the CGRP peptide the C-terminal carboxy moiety is replaced with a moiety selected from: (A) —C(═O)NRR, where R is independently hydrogen, (C₁-C₈)alkyl, haloalkyl, aryl or heteroaryl; and (B) —CH₂OR where R is H, (C₁-C₈)alkyl, aryl or heteroaryl. In some embodiments, this constitutes a carboxy terminally amidated amino acid sequence, such as, but not limited to, a sequence having a C-terminal phenylalaninamide residue or tyrosineamide residue. (See, e.g., Smith et al., Modifications to the N-terminus but not the C-terminus of calcitonin gene-related peptide(8-37) produce antagonists with increased affinity, J. Med. Chem., 46:2427-2435 (2003)).

“Aryl” is phenyl or phenyl vicinally-fused with a saturated, partially-saturated, or unsaturated 3-, 4-, or 5 membered carbon bridge, the phenyl or bridge being substituted by 0, 1, 2 or 3 substituents selected from C₁₈ alkyl, C₁₄ haloalkyl or halo.

“Heteroaryl” is an unsaturated 5, 6 or 7 membered monocyclic or partially-saturated or unsaturated 6-, 7-, 8-, 9-, 10- or 11 membered bicyclic ring, wherein at least one ring is unsaturated, the monocyclic and the bicyclic rings containing 1, 2, 3 or 4 atoms selected from N, O and S, wherein the ring is substituted by 0, 1, 2 or 3 substituents selected from C₁₈ alkyl, C₁₄ haloalkyl and halo.

Although not essential for the practice of the present invention, assay methods for the detection of preferential binding to CGRP₁ receptor are known in the art (e.g., McLatchie et al., Nature, 393:333-339 (1998); Rist et al., J. Med. Chem., 41:117-123 (1998)), and are further exemplified herein below.

A “CGRP peptide antagonist” is a CGRP peptide, such as, but not limited to, a CGRP peptide analog, that antagonizes, blocks, decreases, reduces, impedes, or inhibits CGRP₁ receptor activation by full length native human αCGRP or βCGRP under physiological conditions of temperature, pH, and ionic strength. CGRP peptide antagonists include full and partial antagonists. The present invention does not depend on any particular mechanism of antagonism. For example, the CGRP peptide antagonist can act as a competitive antagonist or a noncompetitive antagonist. Such antagonist activity can be detected by known in vitro methods or in vivo functional assay methods. (See, e.g., Smith et al., Modifications to the N-terminus but not the C-terminus of calcitonin gene-related peptide (8-37) produce antagonists with increased affinity, J. Med. Chem., 46:2427-2435 (2003)). CGRP peptide antagonists lack a “functional CGRP₁ receptor activation” region that is capable of detectably activating a CGRP₁ receptor (or of activating an amylin receptor, adrenomedullin receptor, or CT receptor) at a physiologically or pharmacologically relevant concentration of the peptide. Although not required for the practice of the invention, the skilled artisan is aware of suitable functional assays for detecting CGRP₁ receptor activation, or lack thereof, such as a cAMP-based assay system. In some embodiments the peptide has an amino acid sequence that includes amino acid positions 1-7, or any portion thereof, relative to the native CGRP sequence, however modified, such that the peptide cannot functionally activate the CGRP₁ receptor. Therapeutic administration of CGRP analogs was taught by Evans et al. for the lowering of blood pressure and gastric acid secretion, and for other effects on, for example, ingestion behavior, taste and sensory perception, e.g., nociception. (U.S. Pat. No. 4,530,838; U.S. Pat. No. 4,736,023). Therapeutic use of CGRP antagonists and CGRP-targeting aptamers has been proposed for the treatment of migraine and other disorders. (E.g., Olesen et al., Calcitonin gene-related peptide receptor antagonist BIBN 4096 BS for the acute treatment of migraine, New Engl. J. Med., 350:1104-1110 (2004); Perspective: CGRP-receptor antagonists—a fresh approach to migraine, New Engl. J. Med., 350:1075 (2004); Vater et al., Short bioactive Spiegelmers to migraine-associated calitonin gene-related peptide rapidly identified by a novel approach: tailored-SELEX, Nuc. Acids Res., 31(21 e130):1-7 (2003); WO 96/03993). For example, Noda et al. described the use of CGRP or CGRP derivatives for inhibiting platelet aggregation and for the treatment or prevention of arteriosclerosis or thrombosis. (EP 0385712 B1). Liu et al. disclosed therapeutic agents that modulate the activity of CT receptor, including vehicle-conjugated peptides such as calcitonin and human αCGRP. (WO 01/83526 A2; US 2002/0090646 A1). Vasoactive CGRP peptide antagonists and their use in a method for inhibiting CGRP binding to CGRP receptors were disclosed by Smith et al.; such CGRP peptide antagonists were shown to inhibit CGRP binding to coronary artery membranes and to relax capsaicin-treated pig coronary arteries. (U.S. Pat. No. 6,268,474 B1; and U.S. Pat. No. 6,756,205 B2). Rist et al. disclosed peptide analogs with CGRP receptor antagonist activity and their use in a drug for treatment and prophylaxis of a variety of disorders. (DE 19732944 A1). Example of useful CGRP peptide antagonists include any of those having an amino acid primary structure set forth in Table 2A, Table 2B, Table 2C, Table 2D, Table 3A, Table 3B, Table 3, Table 4, or Table 7 of the U.S. non-provisional application titled CGRP PEPTIDE ANTAGONISTS AND CONJUGATES, U.S. Ser. No. 11/584,177, filed on Oct., 19, 2006, which claims priority from U.S. Provisional Application No. 60/729,083, filed Oct. 21, 2005, both of which are hereby incorporated by reference in their entireties.

A CGRP peptide is comprised of at least 2 to about 90 amino acid residues connected in a main chain by peptide bonds. Amino acid residues are commonly categorized according to different chemical and/or physical characteristics. The term “acidic amino acid residue” refers to amino acid residues in D- or L-form having side chains comprising acidic groups. Exemplary acidic residues include aspartate and glutamate residues. The term “aromatic amino acid residue” refers to amino acid residues in D- or L-form having side chains comprising aromatic groups. Exemplary aromatic residues include tryptophan, tyrosine, 3-(1-naphthyl)alanine, or phenylalanine residues. The term “basic amino acid residue” refers to amino acid residues in D- or L-form having side chains comprising basic groups. Exemplary basic amino acid residues include histidine, lysine, homolysine, ornithine, arginine, N-methyl-arginine, ω-aminoarginine, ω-methyl-arginine, 1-methyl-histidine, 3-methyl-histidine, and homoarginine (hR) residues. The term “hydrophilic amino acid residue” refers to amino acid residues in D- or L-form having side chains comprising polar groups. Exemplary hydrophilic residues include cysteine, serine, threonine, histidine, lysine, asparagine, aspartate, glutamate, glutamine, and citrulline (Cit) residues. The terms “lipophilic amino acid residue” refers to amino acid residues in D- or L-form having sidechains comprising uncharged, aliphatic or aromatic groups. Exemplary lipophilic sidechains include phenylalanine, isoleucine, leucine, methionine, valine, tryptophan, and tyrosine. Alanine (A) is amphiphilic—it is capable of acting as a hydrophilic or lipophilic residue. Alanine, therefore, is included within the definition of both “lipophilic residue” and “hydrophilic residue.” The term “nonfunctional amino acid residue” refers to amino acid residues in D- or L-form having side chains that lack acidic, basic, or aromatic groups. Exemplary neutral amino acid residues include methionine, glycine, alanine, valine, isoleucine, leucine, and norleucine (Nle) residues.

The CGRP peptide includes a first CGRP₁ receptor binding region, or domain, proximal to its carboxy terminal end, which binding region preferentially binds a first binding site on a CRLR-RAMP1 complex. The phrase “proximal to the carboxy terminal end” means close to the peptide's C-terminal amino acid residue (regardless of any lack of a free carboxyl group thereon) by way of the peptide's primary structure, i.e., its amino acid sequence (also known as “primary sequence”), not relating to actual spatial distance or other secondary or higher order structural considerations, or, to whether vehicle is conjugated thereto. Typically, the first CGRP₁ receptor binding region involves the ten amino acid residues most proximal to, and including, the carboxy terminal residue of the peptide, at amino acid positions 28-37 relative to the positional order of the native human αCGRP sequence. However, as long as the peptide retains detectable specific or preferential binding to CRLR-RAMP1 complex, the first CGRP₁ receptor binding region may be 1, 2, 3, 4, or about 5 amino acid residues longer, or 1, 2, or about 3 amino acid residues shorter, than the CGRP₁ receptor binding region at amino acid positions 28-37 of the native human αCGRP sequence.

In some embodiments, the CGRP peptide also includes, from the first CGRP₁ receptor binding region to the N-terminal end of the peptide: a hinge region; and a second CGRP1 receptor binding region between the hinge region and the N-terminal end of the peptide. Thus, the hinge region or domain, is more distal from the carboxy terminal end of the peptide than the first CGRP₁ receptor binding region. The phrase “more distal” means more distant than a certain referent (such as the first CGRP₁ receptor binding region) from the peptide's C-terminal amino acid residue (regardless of any lack of a free carboxyl group thereon) by way of the peptide's primary structure, i.e., its amino acid sequence (or “primary sequence”), not relating to actual spatial distance or other secondary or higher order structural considerations, or to whether vehicle is conjugated thereto. The hinge region provides a movable joint or axis in the peptide that facilitates, participates in, or responds to, CGRP₁ receptor-specific binding by permitting the peptide to bend to better stabilize a peptide-CRLR-RAMP1 complex. Typically, the hinge region involves the nine amino acid residues at amino acid positions 19-27 relative to the positional order of the native human αCGRP sequence. However, a peptide that retains detectable specific or preferential binding to CRLR-RAMP1 complex, can have a hinge region 1, 2, 3, 4, 5, 6, 7, 8, 9 or about 10 amino acid residues longer, or 1, 2, 3, 4, 5, or about 6 amino acid residues shorter, than the hinge region at amino acid positions 19-27 of the native human αCGRP sequence.

Being between the hinge region and the N-terminal end of the peptide, the second CGRP₁ receptor binding region or domain, when present, is located even more distally from the carboxy terminal end than the hinge region. The phrase “even more distal” means at a greater distance than a certain referent (such as the hinge region) from the CGRP peptide's C-terminal amino acid residue (regardless of any lack of a free carboxyl group thereon) by way of the peptide's primary structure, i.e., its amino acid sequence (also known as “primary sequence”), not relating to actual spatial distance or other secondary or higher order structural considerations, or to whether vehicle is conjugated thereto. The second CGRP₁ receptor binding region is involved in increasing the binding affinity of the CGRP peptide either through direct binding to the CRLR-RAMP1 complex and/or via interacting with, or otherwise affecting the conformation of, the first CGRP₁ receptor binding region. Typically, the second CGRP₁ receptor binding region, if present, involves eleven amino acid residues at amino acid positions 8-18 relative to the native human αCGRP sequence. However, a peptide that retains detectable specific binding to CRLR-RAMP1 complex, can have, if present at all, a second CGRP₁ receptor binding region 1, 2, 3, 4, 5, 6, 7, 8, 9, or about 10 amino acid residues shorter than the CGRP₁ receptor binding region at amino acid positions 8-18 of the native human αCGRP sequence ACDTATCVTHRLAGLLSRSGGVVKNNFVPTNVGSKAF-NH₂ (SEQ ID NO:43), or can have a second CGRP₁ receptor binding region longer by 1 or more amino acid residues up to the maximum length of the CGRP peptide, as described herein.

The pharmacologically active peptide, such as but not limited to a CGRP peptide, can be obtained by synthesis employing conventional chemical synthetic methods. For example, solid phase peptide synthesis techniques can be used. Such techniques are well known in the art and include but are not limited to, those described in Merrifield (1973), Chem. Polypeptides, 335-361 (Katsoyannis and Panayotis eds.); Merrifield (1963), J. Am. Chem. Soc., 85:2149; Davis et al. (1985), Biochem. Intl., 10:394-414; Stewart and Young (1969), Solid Phase Peptide Synthesis; U.S. Pat. No. 3,941,763; Finn et al. (1976), The Proteins (3rd ed.) 2:105-253; and Erickson et al., The Proteins (3rd ed.) 2: 257-527 (1976). The use of protecting groups, linkers, and solid phase supports, as well as specific protection and deprotection reaction conditions, linker cleavage conditions, use of scavengers, and other aspects of solid phase peptide synthesis are well known and are also described in “Protecting Groups in Organic Synthesis,” 3rd Edition, T. W. Greene and P. G. M. Wuts, Eds., John Wiley & Sons, Inc., 1999; NovaBiochem Catalog, 2000; “Synthetic Peptides, A User's Guide,” G. A. Grant, Ed., W.H. Freeman & Company, New York, N.Y., 1992; “Advanced Chemtech Handbook of Combinatorial & Solid Phase Organic Chemistry,” W. D. Bennet, J. W. Christensen, L. K. Hamaker, M. L. Peterson, M. R. Rhodes, and H. H. Saneii, Eds., Advanced Chemtech, 1998; “Principles of Peptide Synthesis, 2nd ed.,” M. Bodanszky, Ed., Springer-Verlag, 1993; “The Practice of Peptide Synthesis, 2nd ed.,” M. Bodanszky and A. Bodanszky, Eds., Springer-Verlag, 1994; “Protecting Groups,” P. J. Kocienski, Ed., Georg Thieme Verlag, Stuttgart, Germany, 1994; “Fmoc Solid Phase Peptide Synthesis, A Practical Approach,” W. C. Chan and P. D. White, Eds., Oxford Press, 2000, G. B. Fields et al., Synthetic Peptides: A User's Guide, 1990, 77-183, and elsewhere.

Typically, linear and cyclic peptides are synthesized using Fmoc solid-phase peptide synthesis (SPPS) methodologies on a commercially available synthesizer, such as a Symphony automated synthesizer (Protein Technologies, Inc., Washington, D.C.) or a Liberty microwave assisted automated synthesizer (CEM Corporation, Matthews, N.C.). Protected derivatives of conical amino acids, Fmoc-Dpr(Mtt)-OH, Fmoc-Dab(Mtt)-OH, and Finoc-Cit-OH can be purchased from EMD Biosciences, Inc. (La Jolla, Calif.). Fmoc-homoArg(Pmc)-OH can be purchased from Bachem California, Inc. (Torrance, Calif.). All other non-conical Fmoc-amino acids can be purchased from either Advanced Chemtech (Louisville, Ky.) or Chem-Impex International, Inc. (Wood Dale, Ill.). The coupling reagents 2-(1-H-benzotriazol-1-yl)-1,1,3,3-tetramethyluronium hexafluorophosphate (HBTU) and 1-Benzotriazoyloxytris(pyrrolidino)phosphonium hexafluorophosphate (PyBOP) can be purchased from Matrix Innovation, Inc. (Montreal, Quebec Canada). N-Methyl Pyrrolidone (NMP), dichloromethane (DCM), methanol (MeOH), acetonitrile (ACN), isopropanol, dimethylsulfoxide (DMSO), and anhydrous ethyl ether can be purchased from VWR International (West Chester, Pa.). N,N-dimethylformamide (DMF) is purchased from EMD Biosciences. Trifluoroacetic acid (TFA), N-ethylmorpholine (NEM), pyridine, piperidine, N—N-diisopropylethylamine (DIEA), triisopropylsilane (Tis), phenol, acetic anhydride, and 0.1% TFA in H₂O are purchased from Sigma-Aldrich (St. Louis, Mo.). All solvents and reagents are preferably ACS grade or better and can be used without further purification. Peptides are assembled on CLEAR-amide-MBHA resin (0.44 meq/g substitution), purchased from Peptides International (Louisville, Ky.). Typically, the syntheses are performed using 16 mL polypropylene reaction vessels fitted with course frits (Protein Technologies). Approximately 455 mg resin (0.2 mmole) is added to each reaction vessel and solvated for 10 min in DMF. The growing peptide chains are assembled on the amide-resin starting from the C-terminus using the general amino acid cycle as follows: The N^{α}-Fmoc groups are removed by addition of 5 mL 20% piperidine in DMF for 5 min, followed by a 20 min 5 mL incubation. Amino acids (3-fold molar excess) are added to the resin (3000 μL of 0.2 M amino acid solution in NMP), followed by the addition of 3-fold excess HBTU and 6-fold excess NEM (1.2 mL of 0.5 M HBTU & 1.0 M NEM in DMF). The mixture is agitated by periodic sparging with nitrogen for 45 min, followed by emptying of the reaction vessel by positive nitrogen pressure. The resin is washed with 5 mL of DMF (4×30 sec). A second coupling reaction is repeated for 30 min, the reaction vessel emptied, and the N^{α}-Fmoc-protected peptide-resin is washed with 5 mL DMF (3×30 sec) and 5 mL DCM (2×30 sec). The amino acid coupling cycle is repeated with required amino acids until the desired peptide is assembled. Following N^{α}-Fmoc deprotection of the final amino acid, acetylation of the N^{α}-amine is performed by addition of 2.5 mL acetic anhydride/DIEA solution (1.0 M in DMF) to the reaction vessels and mixed for 30 min. If peptides do not require cyclization, the acetylated peptide-resin is washed with 5 mL DCM (5×30 sec) and dried thoroughly prior to cleavage from the resin and removal of side chain protecting groups. Deprotection of the amino acid side chains and cleavage of the acetylated-peptide from the resin is performed by incubating the peptide-resin with 15 mL cleavage cocktail (92.5% TFA, 2.5% water, 2.5% T is, 2.5% phenol) for 3 hr. The cleavage product is filtered under positive nitrogen gas pressure into tarred 50 mL polypropylene conical tubes. The resin is washed with 10 mL cleavage cocktail for 5 min, filtered, and the filtrates combined. The cleavage solutions are concentrated to approximately 5 mL total volume under a gentle stream of nitrogen. Cold (−20° C.) anhydrous ethyl ether (up to 50 mL) is added to the filtrates. The flocculent peptides are pelleted by centrifugation (e.g., Eppendorf centrifuge 5702 using a swinging bucket rotor) at 3800 rpm for 5 min and the ether is decanted. The peptide pellets are washed with additional cold anhydrous ethyl ether (up to 50 mL), pelleted by centrifugation, decanted, and dried in vacuo. The crude peptide yields typically range from 60% to 99% of the theoretical yields. Crude peptides are dissolved in DMSO and purified by RP-HPLC using, e.g., an Agilent 1100 preparative chromatography system with a photodiode array detector (Agilent Technologies, Inc., Santa Clara, Calif.) and a preparative RP-HPLC bonded silica column (Phenomenex Jupiter C18(2), 300 Å, 10 μM, 50×250 mm) and lyophilized to form amorphous solids. The purified peptides are preferably at least >95% pure as determined by the analytical RP-HPLC using a linear gradient of 2-60% B over 60 min (A=0.1% TFA in H₂O, B=0.1% TFA in CAN, column=Phenomenex Jupiter Proteo, 90 Å, 4 μM, 2.1×50 mm). Correct molecular mass can be confirmed by LC-MS methodologies using, e.g., a Waters Acquity HPLC equipped with a LCT Premier XE orthogonal acceleration time-of-flight (oa-TOF) benchtop mass spectrometer (Waters Corporation, Milford, Mass.). CGRP peptide antagonists, or other pharmacologically active peptides, cyclized with a lactam bridge are prepared by selectively incorporating residues with nucleophilic side chains that will form the lactam protected with 4-methyltrityl (Mtt) and electrophilic side chains with 2-phenyl-isopropyl (2-PhiPr). Amino acids involved in the cyclizations are purchased from EMD Biosciences, Inc. All other amino acids used in the syntheses are preferably standard t-butyl, pentamethyldihydrobenzofuran-5-sulfonyl, or pentamethylchroman-6-sulfonyl side-chain protected Fmoc amino acids. Mtt and 2-PhiPr groups are selectively removed from cyclic CGRP peptide antagonists by addition of 10 mL TFA/T is/DCM (0.3:0.5:9.2) to the peptide-resin (5×10 min), followed by washing the peptide-resin with 10 mL of a 2% DIEA solution (2×2 min) and 10 mL DCM (4×1 min). The lactam bridge in cyclic peptides are formed by activating electrophilic carboxyl groups with 5-fold excess PyBOP and 7-fold excess DIEA in DMF. The mixtures are agitated with continuous sparging with nitrogen. Reactions are monitored by cleavage of a 1-2 mg aliquot of peptide-resin in 400 μL of cleavage cocktail, followed by filtration, concentration of the filtrate under nitrogen, and analyzing by LC-MS methodologies previously described for linear peptides. Lactam formation ranges from 70-99% after approximately 1-4 days at room temperature. The peptide-resin is washed with DMF, acetylated, and washed again with DCM following the cyclization reactions and thoroughly dried in vacuo prior to cleavage. Cyclic peptide-resins are cleaved, purified, and analyzed in the same manner as described for linear peptides. The preceding methods are merely illustrative, and the skilled artisan is aware of various other methods and technical variations for synthesizing the pharmacologically active peptides.

The pharmacologically active peptides can also be obtained using recombinant DNA- and/or RNA-mediated protein expression techniques, or any other methods of preparing peptides or, when applicable, fusion proteins. For example, the peptides can be made in transformed host cells. Briefly, a recombinant DNA molecule coding for the peptide is prepared. Methods of preparing such DNA molecules are well known in the art. For instance, sequences encoding the peptides can be excised from DNA using suitable restriction enzymes. Alternatively, the DNA molecule can be synthesized using chemical synthesis techniques, such as the phosphoramidate method. Also, a combination of these techniques can be used. Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art. Modifications can be made at the DNA level, as well. The peptide-encoding DNA sequence may be changed to codons more compatible with the chosen host cell. For E. coli, optimized codons are known in the art. Codons can be substituted to eliminate restriction sites or to include silent restriction sites, which may aid in processing of the DNA in the selected host cell. Next, the transformed host is cultured and purified. Host cells may be cultured under conventional fermentation conditions so that the desired compounds are expressed. Such fermentation conditions are well known in the art.

Obtaining the pharmacologically active peptide, whether the peptide is prepared by synthetic or recombinant techniques, can also involve suitable protein purification techniques, when applicable. In some embodiments of the PEG-conjugated pharmacologically active peptide, the peptide portion can be prepared to include a suitable isotopic label (e.g., ¹²⁵I, ¹⁴C, ¹³C, ³⁵S, ³H, ²H, ¹³N, ¹⁵N, ¹⁸O, ¹⁷O, etc.), for ease of quantification or detection.

In accordance with the present invention, “obtained” or “obtaining” a peptide means that the peptide is prepared, synthesized, produced, brought into existence, purified, isolated, gotten and/or purchased.

In some useful embodiments of the invention, the pharmacologically active peptide is modified in one or more ways relative to a native sequence of interest, such as but not limited to, a native human αCGRP or βCGRP sequence. The one or more useful modifications can include amino acid additions or insertions, amino acid deletions, peptide truncations, amino acid substitutions, and/or chemical derivatization of amino acid residues, accomplished by known chemical techniques. For example, the thusly modified amino acid sequence includes at least one amino acid residue inserted or substituted therein, relative to the amino acid sequence of the native sequence of interest, in which the inserted or substituted amino acid residue has a side chain comprising a nucleophilic or electrophilic reactive functional group by which the peptide is conjugated to the vehicle. In accordance with the invention, useful examples of such a nucleophilic or electrophilic reactive functional group include, but are not limited to, a thiol, a primary amine, a seleno, a hydrazide, an aldehyde, a carboxylic acid, a ketone, an aminooxy, a masked (protected) aldehyde, or a masked (protected) keto functional group. Examples of amino acid residues having a side chain comprising a nucleophilic reactive functional group include, but are not limited to, a lysine residue, a homolysine, an α,β-diaminopropionic acid residue, an α,γ-diaminobutyric acid residue, an ornithine residue, a cysteine, a homocysteine, a glutamic acid residue, an aspartic acid residue, or a selenocysteine residue.

The useful pharmacologically active peptide according to the present invention can have one or more amino acid additions or insertions, amino acid deletions, peptide truncations, amino acid substitutions, and/or chemical derivatizations of amino acid residues, relative to the sequence of a peptide whose sequence is described herein, so long as the requisite pharmacological activity is maintained.

Examples of useful CGRP peptide antagonists include CGRP peptides having an amino acid primary sequence of any of those set forth in WO 2007/048026 A2 (published Apr. 26, 2007), in Table 2A, Table 2B, Table 2C, Table 2D, Table 3A, Table 3B, Table 3, Table 4, or Table 7, of U.S. Non-provisional patent application Ser. No. 11/584,177, titled “CGRP peptide antagonists and conjugates” by Gegg et al., filed Oct. 19, 2006, which claims the benefit of priority of U.S. Provisional Patent Application No. 60/729,083, filed Oct. 21, 2005, both of which are incorporated herein by reference in their entireties, whether shown in PEG-conjugated or unconjugated form.

For example, additional amino acid residues can be included at the N-terminal end of the CGRP peptide, as long as they do not significantly reduce the potency of CGRP₁ receptor binding or otherwise interfere with CGRP₁ receptor antagonism. For example, in some embodiments, an aromatic amino acid residue, such as a tryptophan or tyrosine residue, can be a useful addition at the N-terminal end of the peptide as a chromophore for quantification or detection purposes, or can, in some embodiments, actually improve potency. Such additions can also be made to N-terminally truncated CGRP peptide analogs as described herein.

In further describing the pharmacologically active peptide herein, a one-letter abbreviation system is frequently applied to designate the identities of the twenty “canonical” amino acid residues generally incorporated into naturally occurring peptides and proteins (Table 1). Such one-letter abbreviations are entirely interchangeable in meaning with three-letter abbreviations, or non-abbreviated amino acid names. Within the one-letter abbreviation system used herein, an upper case letter indicates a L-amino acid, and a lower case letter indicates a D-amino acid. For example, the abbreviation “R” designates L-arginine and the abbreviation “r” designates D-arginine.

TABLE 1
One-letter abbreviations for the canonical amino acids. Three-letter
abbreviations are in parentheses.

| | |
|---|---|
| Alanine (Ala) | A |
| Glutamine (Gln) | Q |
| Leucine (Leu) | L |
| Serine (Ser) | S |
| Arginine (Arg) | R |
| Glutamic Acid (Glu) | E |
| Lysine (Lys) | K |
| Threonine (Thr) | T |
| Asparagine (Asn) | N |
| Glycine (Gly) | G |
| Methionine (Met) | M |
| Tryptophan (Trp) | W |
| Aspartic Acid (Asp) | D |
| Histidine (His) | H |
| Phenylalanine (Phe) | F |
| Tyrosine (Tyr) | Y |
| Cysteine (Cys) | C |
| Isoleucine (Ile) | I |
| Proline (Pro) | P |
| Valine (Val) | V |

An amino acid substitution in an amino acid sequence is typically designated herein with a one-letter abbreviation for the amino acid residue in a particular position, followed by the numerical amino acid position relative to the native sequence of interest (e.g., human αCGRP sequence), which is then followed by the one-letter symbol for the amino acid residue substituted in. For example, “T30D” symbolizes a substitution of a threonine residue by an aspartate residue at amino acid position 30, relative to the native human αCGRP sequence (i.e., ACDTATCVTHRLAGLLSRSGGVVKNNFVPTNVGSKAF-NH₂ HI SEQ ID NO:43), which can serve as a reference sequence of interest.

By way of further example, “R18hR” or “R18Cit” indicates a substitution of an arginine residue by a homoarginine (herein abbreviated “hR” or “hArg”) residue or a citrulline (herein abbreviated “Cit”) residue, respectively, at amino acid position 18, relative to the native human αCGRP sequence (i.e., SEQ ID NO:43). An amino acid position within the amino acid sequence of any particular CGRP peptide (or CGRP peptide analog) described herein may differ from its position relative to the native human αCGRP sequence, i.e., as determined in an alignment of the C-terminal end of the peptide's amino acid sequence with the C-terminal end of the native human αCGRP sequence. For example, amino acid position I of the sequence VTHRLAGLLSRSGGVVKNNFVPTNVGSKAF (SEQ ID NO: 1; human αCGRP(8-37)), thus aligned, corresponds to amino acid position 8 relative to the native human αCGRP reference sequence (SEQ ID NO:43), and amino acid position 30 of SEQ ID NO: 1 corresponds to amino acid position 37 relative to the native human αCGRP reference sequence (SEQ ID NO:43). As a further example, addition of a tryptophan at the N-terminus of a peptide having an amino acid sequence SEQ ID NO: 1 constitutes a “WO addition,” but in relation to the sequence WVTHRLAGLLSRSGGVVKNNFVPTNVGSKAF (SEQ ID NO:4), the tryptophan is at amino acid position 1, while it corresponds to amino acid position 7 relative to the native human αCGRP sequence (SEQ ID NO:43).

In certain embodiments of the present invention, amino acid substitutions encompass, non-canonical amino acid residues, which include naturally rare (in peptides or proteins) amino acid residues or unnatural amino acid residues. Non-canonical amino acid residues can be incorporated into the peptide by chemical peptide synthesis rather than by synthesis in biological systems, such as recombinantly expressing cells, or alternatively the skilled artisan can employ known techniques of protein engineering that use recombinantly expressing cells. (See, e.g., Link et al., Non-canonical amino acids in protein engineering, Current Opinion in Biotechnology, 14(6):603-609 (2003)). The term “non-canonical amino acid residue” refers to amino acid residues in D- or L-form that are not among the 20 canonical amino acids generally incorporated into naturally occurring proteins, for example, β-amino acids, homoamino acids, cyclic amino acids and amino acids with derivatized side chains. Examples include (in the L-form or D-form; abbreviated as in parentheses): citrulline (Cit), homocitrulline (hCit), N^{α}-methylcitrulline (NMeCit), N^{α}-methylhomocitrulline (N^{α}-MeHoCit), ornithine (Orn), N^{α}-Methylornithine (N^{α}-MeOm or NMeOrn), sarcosine (Sar), homolysine (hLys or hK), homoarginine (hArg or hR), homoglutamine (hQ), N^{α}-methylarginine (NMeR), N^{α}-methylleucine (N^{α}-MeL or NMeL), N-methylhomolysine (NMeHoK), N^{α}-methylglutamine (NMeQ), norleucine (Nle), norvaline (Nva), 1,2,3,4-tetrahydroisoquinoline (Tic), Octahydroindole-2-carboxylic acid (Oic), 3-(1-naphthyl)alanine (1-NaI), 3-(2-naphthyl)alanine (2-NaI), 1,2,3,4-tetrahydroisoquinoline (Tic), 2-indanylglycine (Igi), para-iodophenylalanine (pI-Phe), para-aminophenylalanine (4AmP or 4-Amino-Phe), 4-guanidino phenylalanine (Guf), nitrophenylalanine (nitrophe), aminophenylalanine (aminophe or Amino-Phe), benzylphenylalanine (benzylphe), γ-carboxyglutamic acid (γ-carboxyglu), hydroxyproline (hydroxypro), p-carboxyl-phenylalanine (Cpa), α-aminoadipic acid (Aad), Nα-methyl valine (NMeVal), N-α-methyl leucine (NMeLeu), Nα-methylnorleucine (NMeNle), cyclopentylglycine (Cpg), cyclohexylglycine (Chg), acetylarginine (acetylarg), α,β-diaminopropionoic acid (Dpr), α,γ-diaminobutyric acid (Dab), diaminopropionic acid (Dap), cyclohexylalanine (Cha), 4-methyl-phenylalanine (MePhe), β,β-diphenyl-alanine (BiPhA), aminobutyric acid (Abu), 4-phenyl-phenylalanine (or biphenylalanine; 4Bip), α-amino-isobutyric acid (Aib), beta-alanine, beta-aminopropionic acid, piperidinic acid, aminocaprioic acid, aminoheptanoic acid, aminopimelic acid, desmosine, diaminopimelic acid, N-ethylglycine, N-ethylaspargine, hydroxylysine, allo-hydroxylysine, isodesmosine, allo-isoleucine, N-methyl glycine, N-methyl isoleucine, N-methylvaline, 4-hydroxyproline (Hyp), γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-methylarginine, 4-Amino-O-Phthalic Acid (4APA), and other similar amino acids, and derivatized forms of any of these as described herein. Table 1A contains some exemplary non-canonical amino acid residues that are useful in accordance with the present invention and associated abbreviations as typically used herein, although the skilled practitioner will understand that different abbreviations and nomenclatures may be applicable to the same substance and my appear interchangeably herein.

Table 1A. Useful non-canonical amino acids for amino acid addition, insertion, or substitution into CGRP peptide sequences in accordance with the present invention. In the event an abbreviation listed in Table 1A differs from another abbreviation for the same substance disclosed elsewhere herein, both abbreviations are understood to be applicable.

| Abbreviation | Amino Acid |
|---|---|
| Sar | Sarcosine |
| Nle | norleucine |
| Ile | isoleucine |
| 1-Nal | 3-(1-naphthyl)alanine |
| 2-Nal | 3-(2-naphthyl)alanine |
| Bip | 4,4′-biphenyl alanine |
| Dip | 3,3-diphenylalanine |
| Nvl | norvaline |
| NMe-Val | Nα-methyl valine |
| NMe-Leu | Nα-methyl leucine |
| NMe-Nle | Nα-methyl norleucine |
| Cpg | cyclopentyl glycine |
| Chg | cyclohexyl glycine |
| Hyp | hydroxy proline |
| Oic | Octahydroindole-2-Carboxylic |
| | Acid |
| Igl | Indanyl glycine |
| Aib | aminoisobutyric acid |
| Aic | 2-aminoindane-2-carboxylic |
| | acid |
| Pip | pipecolic acid |
| BhTic | β-homo Tic |
| BhPro | β-homo proline |
| Sar | Sarcosine |
| Cpg | cyclopentyl glycine |
| Tiq | 1,2,3,4-L-Tetrahydroisoquinoline-1-Carboxylic |
| | acid |
| Nip | Nipecotic Acid |
| Thz | Thiazolidine-4-carboxylic acid |
| Thi | 3-thienyl alanine |
| 4GuaPr | 4-guanidino proline |
| 4Pip | 4-Amino-1-piperidine-4-carboxylic acid |
| Idc | indoline-2-carboxylic acid |
| Hydroxyl-Tic | 1,2,3,4-Tetrahydroisoquinoline-7-hydroxy-3- |
| | carboxylic acid |
| Bip | 4,4′-biphenyl alanine |
| Ome-Tyr | O-methyl tyrosine |
| I-Tyr | Iodotyrosine |
| Tic | 1,2,3,4-L-Tetrahydroisoquinoline-3-carboxylic |
| | acid |
| Igl | Indanyl glycine |
| BhTic | β-homo Tic |
| BhPhe | β-homo phenylalanine |
| AMeF | α-methyl Phenyalanine |
| BPhe | β-phenylalanine |
| Phg | phenylglycine |
| Anc | 3-amino-2-naphthoic acid |
| Atc | 2-aminotetraline-2-carboxylic acid |
| NMe-Phe | Nα-methyl phenylalanine |
| NMe-Lys | Nα-methyl lysine |
| Tpi | 1,2,3,4-Tetrahydronorharman-3-Carboxylic acid |
| Cpg | cyclopentyl glycine |
| Dip | 3,3-diphenylalanine |
| 4Pal | 4-pyridinylalanine |
| 3Pal | 3-pyridinylalanine |
| 2Pal | 2-pyridinylalanine |
| 4Pip | 4-Amino-1-piperidine-4-carboxylic acid |
| 4AmP | 4-amino-phenylalanine |
| Idc | indoline-2-carboxylic acid |
| Chg | cyclohexyl glycine |
| hPhe | homophenylalanine |
| BhTrp | β-homotryptophan |
| pI-Phe | 4-iodophenylalanine |
| Aic | 2-aminoindane-2-carboxylic acid |
| NMe-Lys | Nα-methyl lysine |
| Orn | ornithine |
| Dpr | 2,3-Diaminopropionic acid |
| Dbu | 2,4-Diaminobutyric acid |
| homoLys | homolysine |
| N-eMe-K | Nε-methyl-lysine |
| N-eEt-K | Nε-ethyl-lysine |
| N-eIPr-K | Nε-isopropyl-lysine |
| bhomoK | β-homolysine |
| rLys | Lys ψ(CH2NH)-reduced amide bond |
| rOrn | Orn ψ(CH2NH)-reduced amide bond |
| Acm | acetamidomethyl |
| Ahx | 6-aminohexanoic acid |
| εAhx | 6-aminohexanoic acid |
| K(NPeg11) | Nε-(O-(aminoethyl)-O′-(2-propanoyl)- |
| | undecaethyleneglycol)-Lysine |
| K(NPeg27) | Nε-(O-(aminoethyl)-O′-(2-propanoyl)- |
| | (ethyleneglycol)27-Lysine |
| Cit | Citrulline |
| hArg | homoarginine |
| hCit | homocitrulline |
| NMe-Arg | Nα-methyl arginine (NMeR) |
| Guf | 4-guanidinyl phenylalanine |
| bhArg | β-homoarginine |
| 3G-Dpr | 2-amino-3-guanidinopropanoic acid |
| 4AmP | 4-amino-phenylalanine |
| 4AmPhe | 4-amidino-phenylalanine |
| 4AmPig | 2-amino-2-(1-carbamimidoylpiperidin-4- |
| | yl)acetic acid |
| 4GuaPr | 4-guanidino proline |
| N-Arg | Nα-[(CH₂)₃NHCH(NH)NH₂] substituted glycine |
| rArg | Arg ψ(CH2NH)-reduced amide bond |
| 4PipA | 4-Piperidinyl alanine |
| NMe-Arg | Nα-methyl arginine (or NMeR) |
| NMe-Thr | Nα-methyl threonine(or NMeThr) |

Nomenclature and Symbolism for Amino Acids and Peptides by the UPAC-IUB Joint Commission on Biochemical Nomenclature (JCBN) have been published in the following documents: Biochem. J., 1984, 219, 345-373; Eur. J. Biochem., 1984, 138, 9-37; 1985, 152, 1; 1993, 213, 2; Internat. J. Pept. Prot. Res., 1984, 24, following p 84; J. Biol. Chem., 1985, 260, 14-42; Pure Appl. Chem., 1984, 56, 595-624; Amino Acids and Peptides, 1985, 16, 387-410; Biochemical Nomenclature and Related Documents, 2nd edition, Portland Press, 1992, pages 39-69.

In some embodiments, the amino acid sequence of the pharmacologically active peptide is modified, relative to a native reference sequence of interest, such that the modification reduces the peptide's susceptibility to enzymatic proteolysis. For example, for CGRP peptides, the reference sequence can be a human αCGRP reference sequence (SEQ ID NO:43). This reduced susceptibility may be due to an effect on a protease binding site or cleavage site for an exopeptidase or endopeptidase. Some native peptides of interest are rapidly degraded in biological fluid by proteolytic enzymes such as serine proteases (trypsin, chymotrypsin, and elastase). For example, the half-life of CGRP in mammalian plasma is reported to be about 10 min [A. S. Thakor, D. A. Giussani, Circulation. 2005; 112:2510-2516; Kraenzlin M E, Ch'ng J L, Mulderry P K, Ghatei M A, Bloom S R., Regul Pept. 1985 March; 10(2-3):189-97.]. Proteolytic enzymes have varying substrate specificities, for instance, trypsin, preferentially cleaves at Arg and Lys in position P1 with higher rates for Arg. [Keil, B. Specificity of proteolysis. Springer-Verlag Berlin-Fleidelberg-New York, pp. 335. (1992)]. On this basis multiple sites within a given peptide or protein can be recognized and processed by different proteases.

Examples of modification to the native sequence of a pharmacologically active peptide of interest are the replacement of arginine (R) with citrulline, and phenylalanine (F) with 3-(1-naphthyl)alanine. The introduction of non-canonical or unnatural amino acids and cyclic structures has been carried out in an iterative manner to identify novel CGRP analogues with significantly improved stability in biological fluid (e.g., 100% plasma) with or without PEGylation (20 kDa MeO-PEG), as illustrated in Example 2 of U.S. Non-provisional patent application Ser. No. 11/584,177, titled “CGRP peptide antagonists and conjugates” by Gegg et al., filed Oct. 19, 2006, which claims the benefit of priority of U.S. Provisional Patent Application No. 60/729,083, filed Oct. 21, 2005, both of which are incorporated herein by reference in their entireties.

The term “protease” is synonymous with “peptidase”. Proteases comprise two groups of enzymes: the endopeptidases which cleave peptide bonds at points within the protein, and the exopeptidases, which remove one or more amino acids from either N- or C-terminus respectively. The term “proteinase” is also used as a synonym for endopeptidase. The four mechanistic classes of proteinases are: serine proteinases, cysteine proteinases, aspartic proteinases, and metallo-proteinases. In addition to these four mechanistic classes, there is a section of the enzyme nomenclature which is allocated for proteases of unidentified catalytic mechanism. This indicates that the catalytic mechanism has not been identified.

Cleavage subsite nomenclature is commonly adopted from a scheme created by Schechter and Berger (Schechter I. & Berger A., On the size of the active site in proteases. I. Papain, Biochemical and Biophysical Research Communication, 27:157 (1967); Schechter 1. & Berger A., On the active site of proteases. 3. Mapping the active site of papain; specific peptide inhibitors of papain, Biochemical and Biophysical Research Communication, 32:898 (1968)). According to this model, amino acid residues in a substrate undergoing cleavage are designated P1, P2, P3, P4 etc. in the N-terminal direction from the cleaved bond. Likewise, the residues in the C-terminal direction are designated P1′, P2′, P3′, P4′. etc.

The skilled artisan is aware of a variety of tools for identifying protease binding or protease cleavage sites of interest. For example, the PeptideCutter software tool is available through the ExPASy (Expert Protein Analysis System) proteomics server of the Swiss Institute of Bioinformatics (SIB; www.expasy.org/tools/peptidecutter). PeptideCutter searches a protein sequence from the SWISS-PROT and/or TrEMBL databases or a user-entered protein sequence for protease cleavage sites. Single proteases and chemicals, a selection or the whole list of proteases and chemicals can be used. Different forms of output of the results are available: Tables of cleavage sites either grouped alphabetically according to enzyme names or sequentially according to the amino acid number. A third option for output is a map of cleavage sites. The sequence and the cleavage sites mapped onto it are grouped in blocks, the size of which can be chosen by the user. Other tools are also known for determining protease cleavage sites. (E.g., Turk, B. et al., Determination of protease cleavage site motifs using mixture-based oriented peptide libraries, Nature Biotechnology, 19:661-667 (2001); Barrett A. et al., Handbook of proteolytic enzymes, Academic Press (1998)).

The serine proteinases include the chymotrypsin family, which includes mammalian protease enzymes such as chymotrypsin, trypsin or elastase or kallikrein. The serine proteinases exhibit different substrate specificities, which are related to amino acid substitutions in the various enzyme subsites interacting with the substrate residues. Some enzymes have an extended interaction site with the substrate whereas others have a specificity restricted to the P1 substrate residue.

Trypsin preferentially cleaves at R or K in position P1. A statistical study carried out by Keil (1992) described the negative influences of residues surrounding the Arg- and Lys-bonds (i.e. the positions P2 and P1′, respectively) during trypsin cleavage. (Keil, B., Specificity of proteolysis, Springer-Verlag Berlin-Heidelberg-New York, 335 (1992)). A proline residue in position P1′ normally exerts a strong negative influence on trypsin cleavage. Similarly, the positioning of R and K in P1′ results in an inhibition, as well as negatively charged residues in positions P2 and P1′.

Chymotrypsin preferentially cleaves at a W, Y or F in position P1 (high specificity) and to a lesser extent at L, M or H residue in position P1. (Keil, 1992). Exceptions to these rules are the following: When W is found in position P1, the cleavage is blocked when M or P are found in position P1′ at the same time. Furthermore, a proline residue in position P1′ nearly fully blocks the cleavage independent of the amino acids found in position P1. When an M residue is found in position P1, the cleavage is blocked by the presence of a Y residue in position P1′. Finally, when H is located in position P1, the presence of a D, M or W residue also blocks the cleavage.

Membrane metallo-endopeptidase (NEP; neutral endopeptidase, kidney-brush-border neutral proteinase, enkephalinase, EC 3.4.24.11) cleaves peptides at the amino side of hydrophobic amino acid residues. (Connelly, J C et al., Neutral Endopeptidase 24.11 in Human Neutrophils Cleavage of Chemotactic Peptide, PNAS, 82(24):8737-8741 (1985)).

Thrombin preferentially cleaves at an R residue in position P1. (Keil, 1992). The natural substrate of thrombin is fibrinogen. Optimum cleavage sites are when an R residue is in position P1 and Gly is in position P2 and position P1′. Likewise, when hydrophobic amino acid residues are found in position P4 and position P3, a proline residue in position P2, an R residue in position P1, and non-acidic amino acid residues in position P1′ and position P2′. A very important residue for its natural substrate fibrinogen is a D residue in P10.

Caspases are a family of cysteine proteases bearing an active site with a conserved amino acid sequence and which cleave peptides specifically following D residues. (Earnshaw W C et al., Mammalian caspases: Structure, activation, substrates, and functions during apoptosis, Annual Review of Biochemistry, 68:383-424 (1999)).

The Arg-C proteinase preferentially cleaves at an R residue in position P1. The cleavage behavior seems to be only moderately affected by residues in position P1′. (Keil, 1992). The Asp-N endopeptidase cleaves specifically bonds with a D residue in position P1′. (Keil, 1992).

Furin is a ubiquitous subtilisin-like proprotein convertase. It is the major processing enzyme of the secretory pathway and intracellularly is localized in the trans-golgi network (van den Ouweland, A. M. W. et al. (1990) Nucl. Acids Res., 18, 664; Steiner, D. F. (1998) Curr. Opin. Chem. Biol., 2, 31-39). The minimal furin cleavage site is Arg-X-X-Arg′. However, the enzyme prefers the site Arg-X-(Lys/Arg)-Arg′. An additional arginine at the P6 position appears to enhance cleavage (Krysan, D. J. et al. (1999) J. Biol. Chem., 274, 23229-23234).

The foregoing is merely exemplary and by no means an exhaustive treatment of knowledge available to the skilled artisan concerning protease binding and/or cleavage sites that the skilled artisan may be interested in eliminating in practicing the invention.

Additional useful embodiments of PEG-conjugated pharmacologically active peptides can result from conservative modifications of the amino acid sequences of the pharmacologically active peptides of interest. Conservative modifications will produce PEG-conjugated peptides having functional, physical, and chemical characteristics similar to those of the PEG-conjugated peptide from which such modifications are made. Such conservatively modified forms of the PEG-conjugated peptides disclosed herein are also contemplated as being in accordance with the present invention.

In contrast, substantial modifications in the functional and/or chemical characteristics of the peptides may be accomplished by selecting substitutions in the amino acid sequence that differ significantly in their effect on maintaining (a) the structure of the molecular backbone in the region of the substitution, for example, as an α-helical conformation, (b) the charge or hydrophobicity of the molecule at the target site, or (c) the size of the molecule.

For example, a “conservative amino acid substitution” may involve a substitution of a native amino acid residue with a normative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., Acta Physiol. Scand. Suppl., 643:55-67 (1998); Sasaki et al., 1998, Adv. Biophys. 35:1-24 (1998), which discuss alanine scanning mutagenesis).

Desired amino acid substitutions (whether conservative or non-conservative) can be determined by those skilled in the art at the time such substitutions are desired. For example, amino acid substitutions can be used to identify important residues of the peptide sequence, or to increase or decrease the affinity of the peptide or vehicle-conjugated peptide molecules described herein.

Naturally occurring residues may be divided into classes based on common side chain properties:

1) hydrophobic: norleucine (Nor), Met, Ala, Val, Leu, Ile;

2) neutral hydrophilic: Cys, Ser, Thr, Asn, Gln;

3) acidic: Asp, Glu;

4) basic: His, Lys, Arg;

5) residues that influence chain orientation: Gly, Pro; and

6) aromatic: Trp, Tyr, Phe.

Conservative amino acid substitutions may involve exchange of a member of one of these classes with another member of the same class. Conservative amino acid substitutions may encompass non-naturally occurring amino acid residues, which are typically incorporated by chemical peptide synthesis rather than by synthesis in biological systems. These include peptidomimetics and other reversed or inverted forms of amino acid moieties.

Non-conservative substitutions may involve the exchange of a member of one of these classes for a member from another class. Such substituted residues may be introduced into regions of the human antibody that are homologous with non-human antibodies, or into the non-homologous regions of the molecule.

In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those that are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as disclosed herein. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those that are within ±1 are included, and in certain embodiments, those within ±0.5 are included. One may also identify epitopes from primary amino acid sequences on the basis of hydrophilicity. These regions are also referred to as “epitopic core regions.”

Examples of conservative substitutions include the substitution of one non-polar (hydrophobic) amino acid residue such as isoleucine, valine, leucine norleucine, alanine, or methionine for another, the substitution of one polar (hydrophilic) amino acid residue for another such as between arginine and lysine, between glutamine and asparagine, between glycine and serine, the substitution of one basic amino acid residue such as lysine, arginine or histidine for another, or the substitution of one acidic residue, such as aspartic acid or glutamic acid for another. The phrase “conservative amino acid substitution” also includes the use of a chemically derivatized residue in place of a non-derivatized residue, provided that such polypeptide displays the requisite CGRP₁ receptor antagonist activity. Other exemplary amino acid substitutions that can be useful in accordance with the present invention are set forth in Table 2.

TABLE 2
Some Useful Amino Acid Substitutions.

| Original | Exemplary |
|---|---|
| Residues | Substitutions |
| Ala | Val, Leu, Ile |
| Arg | Lys, Gln, Asn |
| Asn | Gln |
| Asp | Glu |
| Cys | Ser, Ala |
| Gln | Asn |
| Glu | Asp |
| Gly | Pro, Ala |
| His | Asn, Gln, Lys, Arg |
| Ile | Leu, Val, Met, Ala, |
| | Phe, Norleucine |
| Leu | Norleucine, Ile, Val, |
| | Met, Ala, Phe |
| Lys | Arg, 1,4-Diamino- |
| | butyric Acid, Gln, |
| | Asn |
| Met | Leu, Phe, Ile |
| Phe | Leu, Val, Ile, Ala, |
| | Tyr |
| Pro | Ala |
| Ser | Thr, Ala, Cys |
| Thr | Ser |
| Trp | Tyr, Phe |
| Tyr | Trp, Phe, Thr, Ser |
| Val | Ile, Met, Leu, Phe, |
| | Ala, Norleucine |

As stated herein above, in accordance with the present invention, the peptide can also be chemically derivatized at one or more amino acid residues. Peptides that contain derivatized amino acid residues can be synthesized by known organic chemistry techniques. “Chemical derivative” or “chemically derivatized” refers to a subject peptide having one or more residues chemically derivatized by reaction of a functional side group. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides which contain one or more naturally occurring amino acid derivatives of the twenty canonical amino acids, whether in L- or D-form. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

Useful derivatizations include, in some embodiments, those in which the amino terminal of the peptide is chemically blocked so that conjugation with the vehicle will be prevented from taking place at an N-terminal free amino group. There may also be other beneficial effects of such a modification, for example a reduction in the CGRP peptide's susceptibility to enzymatic proteolysis. The N-terminus can be acylated or modified to a substituted amine, or derivatized with another functional group, such as an aromatic moiety (e.g., an indole acid, benzyl (Bzl or Bn), dibenzyl (DiBzl or Bn₂), or benzyloxycarbonyl (Cbz or Z)), N,N-dimethylglycine or creatine. For example, in some embodiments, an acyl moiety, such as, but not limited to, a formyl, acetyl (Ac), propanoyl, butanyl, heptanyl, hexanoyl, octanoyl, or nonanoyl, can be covalently linked to the N-terminal end of the peptide, which can prevent undesired side reactions during conjugation of the vehicle to the peptide. Other exemplary N-terminal derivative groups include —NRR¹ (other than —NH₂), —NRC(O)R¹, —NRC(O)OR¹, —NRS(O)₂R′, —NHC(O)NHR¹, succinimide, or benzyloxycarbonyl-NH— (Cbz-NH—), wherein R and R¹ are each independently hydrogen or lower alkyl and wherein the phenyl ring may be substituted with 1 to 3 substituents selected from C₁-C₄ alkyl, C₁-C₄ alkoxy, chloro, and bromo.

In some embodiments, one or more peptidyl [—C(O)NR—] linkages (bonds) between amino acid residues can be replaced by a non-peptidyl linkage. Exemplary non-peptidyl linkages are —CH₂-carbamate [—CH₂—OC(O)NR—], phosphonate, —CH₂-sulfonamide [—CH₂—S(O)₂NR—], urea [—NHC(O)NH—], —CH₂-secondary amine, and alkylated peptide [—C(O)NR⁶— wherein R⁶ is lower alkyl].

In some embodiments, one or more individual amino acid residues can be derivatized. Various derivatizing agents are known to react specifically with selected sidechains or terminal residues, as described in detail below by way of example.

Lysinyl residues and amino terminal residues may be reacted with succinic or other carboxylic acid anhydrides, which reverse the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues may be modified by reaction with any one or combination of several conventional reagents, including phenylglyoxal, 2,3-butanedione, 1,2-cyclohexanedione, and ninhydrin. Derivatization of arginyl residues requires that the reaction be performed in alkaline conditions because of the high pKa of the guanidine functional group. Furthermore, these reagents may react with the groups of lysine as well as the arginine epsilon-amino group.

Specific modification of tyrosyl residues has been studied extensively, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively.

Carboxyl sidechain groups (aspartyl or glutamyl) may be selectively modified by reaction with carbodiimides (R′—N═C═N—R′) such as 1-cyclohexyl-3-(2-morpholinyl-(4-ethyl)carbodiimide or 1-ethyl-3-(4-azonia-4,4-dimethylpentyl) carbodiimide. Furthermore, aspartyl and glutamyl residues may be converted to asparaginyl and glutaminyl residues by reaction with ammonium ions.

Glutaminyl and asparaginyl residues may be deamidated to the corresponding glutamyl and aspartyl residues. Alternatively, these residues are deamidated under mildly acidic conditions. Either form of these residues falls within the scope of this invention.

Cysteinyl residues can be replaced by amino acid residues or other moieties either to eliminate disulfide bonding or, conversely, to stabilize cross-linking. (See, e.g., Bhatnagar et al., J. Med. Chem., 39:3814-3819 (1996)).

Derivatization with bifunctional agents is useful for cross-linking the peptides or their functional derivatives to a water-insoluble support matrix, if desired, or to other macromolecular vehicles. Commonly used cross-linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates, e.g., as described in U.S. Pat. Nos. 3,969,287; 3,691,016; 4,195,128; 4,247,642; 4,229,537; and 4,330,440, are employed for protein immobilization.

Other possible modifications include hydroxylation of proline and lysine, phosphorylation of hydroxyl groups of seryl or threonyl residues, oxidation of the sulfur atom in Cys, methylation of the alpha-amino groups of lysine, arginine, and histidine side chains. Creighton, Proteins: Structure and Molecule Properties (W. H. Freeman & Co., San Francisco), 79-86 (1983).

The above examples of derivatizations are not intended to be an exhaustive treatment, but merely illustrative.

In some preferred embodiments of the present invention, the pharmacologically active peptide includes at its N-terminal an acyl, acetyl (Ac), benzoyl, benzyloxycarbonyl (Cbz or Z), benzyl (Bzl or Bn), or dibenzyl (DiBzl or Bn₂), or moiety.

The pharmacologically active peptide is conjugated to a polyethylene glycol (PEG) at: (a) 1, 2, 3, or 4 amino functionalized sites of the pharmacologically active peptide.

“PEG-conjugated” means that the pharmacologically active peptide and the PEG are covalently attached or linked to each other, either directly attached, or indirectly attached via a linker moiety.

In being conjugated in accordance with the inventive method, the polyethylene glycol (PEG), as described herein, is covalently bound by reductive amination directly to at least one solvent-exposed free amine moiety of an amino acid residue of the pharmacologically active peptide itself. In some embodiments of the inventive method, the pharmacologically active peptide is conjugated to a PEG at one or more primary or secondary amines on the pharmacologically active peptide, or to two PEG groups at a single primary amine site on the pharmacologically active peptide (e.g., this can occur when the reductive amination reaction involves the presence of excess PEG-aldehyde compound). We have observed that when PEGylation by reductive amination is at a primary amine on the peptide, it is not uncommon to have amounts (1 to 100% range) of reaction product that have two or more PEGs present per molecule, and if the desired PEGylation product is one with only one PEG per molecule, then this “over-PEGylation” may be undesirable. When PEGylated product with a single PEG per PEGylation product molecule is desired, an embodiment of the inventive method can be employed that involves PEGylation using secondary amines of the pharmacologically active peptide, because only one PEG group per molecule will be transferred in the reductive amination reaction.

Amino acid residues that can provide a primary amine moiety include residues of lysine, homolysine, ornithine, α,β-diaminopropionic acid (Dap), α,β-diaminopropionoic acid (Dpr), and α,γ-diaminobutyric acid (Dab), aminobutyric acid (Abu), and α-amino-isobutyric acid (Aib). Amino acid residues that can provide a secondary amine moiety include ε-N-alkyl lysine, α-N-alkyl lysine, δ-N-alkyl ornithine, α-N-alkyl ornithine, or an N-terminal proline, where the alkyl is C₁ to C₆.

Alternatively, the PEG is covalently conjugated to the pharmacologically active peptide indirectly by reductive amination via a solvent-exposed free amine moiety of a peptidyl or non-peptidyl linker (including but not limited to aryl linkers) bearing a free amine moiety exposed for reductive amination with PEG, which linker is covalently bound to an amino acid residue of the pharmacologically active peptide.

Any “linker” group is optional. When present, its chemical structure is not critical, since it serves primarily as a spacer, which can be useful in optimizing pharmacological activity of some embodiments of the inventive composition. The linker is preferably made up of amino acids linked together by peptide bonds. The linker moiety, if present, can be independently the same or different from any other linker, or linkers, that may be present in the inventive composition.

As stated above, the linker, if present, can be peptidyl in nature (i.e., made up of amino acids linked together by peptide bonds) and made up in length, preferably, of from 1 up to about 40 amino acid residues, more preferably, of from 1 up to about 20 amino acid residues, and most preferably of from 1 to about 9 amino acid residues. Preferably, but not necessarily, the amino acid residues in the linker are from among the twenty canonical amino acids, more preferably, cysteine, glycine, alanine, proline, arginine, asparagine, glutamine, serine and/or lysine. Even more preferably, a peptidyl linker is made LIP of a majority of amino acids that are sterically unhindered, such as glycine and alanine linked by a peptide bond. It is also desirable that, if present, a peptidyl linker be selected that avoids rapid proteolytic turnover in circulation in vivo, and/or which includes one or more copies of a binding region of interest, such as a CGRP₁ binding region. Some of these amino acids may be glycosylated, as is well understood by those in the art. For example, a useful linker sequence constituting a sialylation site is X₁X₂NX₄X₅G (SEQ ID NO:50), wherein X₁, X₂, X₄ and X₅ are each independently any amino acid residue.

In other embodiments, the 1 to 40 amino acids are selected from glycine, alanine, proline, asparagine, glutamine, and lysine. Preferably, a linker is made up of a majority of amino acids that are sterically unhindered, such as glycine and alanine. Thus, preferred linkers include polyglycines, poly(Gly-Ala)s, and polyalanines. Some exemplary peptidyl linkers are poly(Gly)₁₋₈, particularly (Gly)₃, (Gly)₄ (SEQ ID NO:51), (Gly)₅ (SEQ ID NO:52) and (Gly)₇ (SEQ ID NO:53), as well as, poly(Gly)₄Ser (SEQ ID NO:54), poly(Gly-Ala)₂₋₄ and poly(Ala)₁₋₈. Other specific examples of peptidyl linkers include (Gly)₅Lys (SEQ ID NO:55), and (Gly)₅LysArg (SEQ ID NO:56). Other specific examples of linkers are: Other examples of useful peptidyl linkers are:

| | |
|---|---|
| (Gly)₃Lys(Gly)₄; | (SEQ ID NO: 57) |
| (Gly)₃AsnGlySer(Gly)₂; | (SEQ ID NO: 58) |
| (Gly)₃Cys(Gly)₄; | (SEQ ID NO: 59) |
| and | |
| GlyProAsnGlyGly. | (SEQ ID NO: 60) |

To explain the above nomenclature, for example, (Gly)₃Lys(Gly)₄ means Gly-Gly-Gly-Lys-Gly-Gly-Gly-Gly (SEQ ID NO:61). Other combinations of Gly and Ala are also useful.

Other preferred linkers are those identified herein as “L5” (GGGGS; SEQ ID NO:62), “L10” (GGGGSGGGGS; SEQ ID NO:63), “L25” GGGGSGGGGSGGGGSGGGGSGGGGS; SEQ ID NO:64) and any linkers used in the working examples hereinafter.

In some embodiments of the compositions of this invention, which comprise a peptide linker moiety (L), acidic residues, for example, glutamate or aspartate residues, are placed in the amino acid sequence of the linker moiety (L). Examples include the following peptide linker sequences:

| | |
|---|---|
| GGEGGG; | (SEQ ID NO: 65) |
| GGEEEGGG; | (SEQ ID NO: 66) |
| GEEEG; | (SEQ ID NO: 67) |
| GEEE; | (SEQ ID NO: 68) |
| GGDGGG; | (SEQ ID NO: 69) |
| GGDDDGG; | (SEQ ID NO: 70) |
| GDDDG; | (SEQ ID NO: 71) |
| GDDD; | (SEQ ID NO: 72) |
| GGGGSDDSDEGSDGEDGGGGS; | (SEQ ID NO: 73) |
| WEWEW; | (SEQ ID NO: 74) |
| FEFEF; | (SEQ ID NO: 75) |
| EEEWWW; | (SEQ ID NO: 76) |
| EEEFFF; | (SEQ ID NO: 77) |
| WWEEEWW; | (SEQ ID NO: 78) |
| or | |
| FFEEEFF. | (SEQ ID NO: 79) |

In other embodiments, the linker constitutes a phosphorylation site, e.g., X₁X₂YX₄X₅G (SEQ ID NO:80), wherein X₁, X₂, X₄ and X₅ are each independently any amino acid residue; X₁X₂SX₄X₅G (SEQ ID NO:81), wherein X₁, X₂, X₄ and X₅ are each independently any amino acid residue; or X₁X₂TX₄X₅G (SEQ ID NO:82), wherein X₁, X₂, X₄ and X₅ are each independently any amino acid residue.

The linkers shown here are exemplary; peptidyl linkers within the scope of this invention may be much longer and may include other residues. A peptidyl linker can contain, e.g., a N-terminal cysteine, another thiol, or nucleophile for conjugation with a vehicle. In another embodiment, the linker contains an N-terminal cysteine or homocysteine residue, or other 2-amino-ethanethiol or 3-amino-propanethiol moiety for conjugation to maleimide, iodoacetaamide or thioester, functionalized vehicles. Another useful peptidyl linker is a large, flexible linker comprising a random Gly/Ser/Thr sequence, for example: GSGSATGGSGSTASSGSGSATH (SEQ ID NO:83) or HGSGSATGGSGSTASSGSGSAT (SEQ ID NO:84), that is estimated to be about the size of a 1 kDa PEG molecule. Alternatively, a useful peptidyl linker may be comprised of amino acid sequences known in the art to form rigid helical structures (e.g., Rigid linker: -AEAAAKEAAAKEAAAKAGG-) (SEQ ID NO:85). Additionally, a peptidyl linker can also comprise a non-peptidyl segment such as a 6 carbon aliphatic molecule of the formula —CH₂—CH₂—CH₂—CH₂—CH₂—CH₂—. The peptidyl linkers can be altered to form derivatives as described herein.

Optionally, non-peptidyl linkers are also useful for conjugating the vehicle to the peptide portion of the vehicle-conjugated CGRP peptide antagonist. For example, alkyl linkers such as —NH—(CH₂)ₛ—C(O)—, wherein s=2-20 can be used. These alkyl linkers may further be substituted by any non-sterically hindering group such as lower alkyl (e.g., C₁-C₆) lower acyl, halogen (e.g., Cl, Br), CN, NH₂, phenyl, etc. Exemplary non-peptidyl linkers are PEG linkers (e.g., shown below):

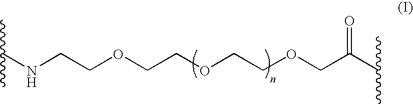

wherein n is such that the linker has a molecular weight of about 100 to about 5000 kilodaltons (kDa), preferably about 100 to about 500 kDa.

In one embodiment, the non-peptidyl linker is aryl. The linkers may be altered to form derivatives in the same manner as described herein. In addition, PEG moieties may be attached to the N-terminal amine or selected side chain amines by either reductive alkylation using PEG aldehydes or acylation using hydroxysuccinimido or carbonate esters of PEG, or by thiol conjugation.

Non-peptide portions of a substance, such as non-peptidyl linkers (or PEG) can be synthesized by conventional organic chemistry reactions familiar to the skilled artisan.

The above is merely illustrative and not an exhaustive treatment of the kinds of linkers that can optionally be employed in accordance with the present invention.

In some useful embodiment of the inventive method of producing a composition of matter, the pharmacologically active peptide is conjugated to a pharmaceutically acceptable PEG at the amino acid residue at the peptide's amino terminal end. (See, e.g., Kinstler et al., N-terminally chemically modified protein compositions and methods, U.S. Pat. Nos. 5,985,265, and 5,824,784).

It will be appreciated that, since the PEG employed for conjugation to the pharmacologically active peptide can be multivalent (e.g., bivalent, trivalent, tetravalent or a higher order valency), as to the number of residues at which PEG can be conjugated, and/or the peptide portion can be multivalent (e.g., bivalent, trivalent, tetravalent or a higher order valency), it is possible by the inventive method of producing a composition of matter to produce a variety of conjugated PEG:peptide structures. By way of example, a univalent PEG and a univalent peptide will produce a 1:1 conjugate; a bivalent peptide and a univalent PEG may form conjugates wherein the peptide conjugates bear two vehicle moieties, whereas a bivalent PEG and a univalent peptide may produce species where two peptide entities are linked to a single PEG moiety; use of higher-valence PEG can lead to the formation of clusters of peptide entities bound to a single PEG moiety, whereas higher-valence peptides may become encrusted with a plurality of PEG moieties.

The peptide moieties may have more than one reactive group which will react with the activated PEG and the possibility of forming complex structures must always be considered; when it is desired to form simple structures such as 1:1 adducts of vehicle and peptide, or to use bivalent vehicles to form peptide:PEG:peptide adducts, it will be beneficial to use predetermined ratios of activated vehicle and peptide material, predetermined concentrations thereof and to conduct the reaction under predetermined conditions (such as duration, temperature, pH, etc.) so as to form a proportion of the described product and then to separate the described product from the other reaction products. The reaction conditions, proportions and concentrations of the reagents can be obtained by relatively simple trial-and-error experiments which are within the ability of an ordinarily skilled artisan with appropriate scaling-up as necessary. Purification and separation of the products is similarly achieved by conventional techniques well known to those skilled in the art.

Additionally, physiologically acceptable salts of the PEG-conjugated pharmacologically active peptide of this invention are also encompassed within the present invention. By “physiologically acceptable salts” is meant any salts that are known or later discovered to be pharmaceutically acceptable. Some specific examples are: acetate; trifluoroacetate; hydrohalides, such as hydrochloride and hydrobromide; sulfate; citrate; maleate; tartrate; glycolate; gluconate; succinate; mesylate; besylate; and oxalate salts.

By “PEGylated peptide” is meant a peptide having a pharmaceutically acceptable polyethylene glycol (PEG) moiety covalently bound to an amino acid residue of the peptide itself or to a peptidyl or non-peptidyl linker (including but not limited to aromatic linkers) that is covalently bound to a residue of the peptide.

By “polyethylene glycol” or “PEG” is meant a polyalkylene glycol compound or a derivative thereof, with or without coupling agents or derivatization with coupling or activating moieties (e.g., with aldehyde, hydroxysuccinimidyl, hydrazide, thiol, triflate, tresylate, azirdine, oxirane, orthopyridyl disulphide, vinylsulfone, iodoacetamide or a maleimide moiety). In accordance with the present invention, useful PEG includes substantially linear, straight chain PEG, branched PEG, or dendritic PEG. (See, e.g., Merrill, U.S. Pat. No. 5,171,264; Harris et al., Multiarmed, monofunctional, polymer for coupling to molecules and surfaces, U.S. Pat. No. 5,932,462; Shen, N-maleimidyl polymer derivatives, U.S. Pat. No. 6,602,498).

PEG is a well-known, water soluble polymer that is commercially available or can be prepared by ring-opening polymerization of ethylene glycol according to methods well known in the art (Sandier and Karo, Polymer Synthesis, Academic Press, New York, Vol. 3, pages 138-161). In the present application, the term “PEG” is used broadly to encompass any polyethylene glycol molecule, in mono-, bi-, or poly-functional form, without regard to size or to modification at an end of the PEG, and can be represented by the formula:

X—O(CH₂CH₂O)ₙ₋₁CH₂CH₂OH, (II)

where n is 20 to 2300 and X is H or a terminal modification, e.g., a C₁₋₄ alkyl.

In some useful embodiments, a PEG used in the invention terminates on one end with hydroxy or methoxy, i.e., X is H or CH₃ (“methoxy PEG”). It is noted that the other end of the PEG, which is shown in formula (II) terminating in OH, covalently attaches to an activating moiety via an ether oxygen bond, an amine linkage, or amide linkage. When used in a chemical structure, the term “PEG” includes the formula (II) above without the hydrogen of the hydroxyl group shown, leaving the oxygen available to react with a free carbon atom of a linker to form an ether bond. More specifically, in order to conjugate PEG to a peptide, the peptide must be reacted with PEG in an “activated” form. Activated PEG can be represented by the formula:

(PEG)-(A) (III)

where PEG (defined supra) covalently attaches to a carbon atom of the activation moiety (A) to form an ether bond, an amine linkage, or amide linkage, and (A) contains a reactive group which can react with an amino, imino, or thiol group on an amino acid residue of a peptide or a linker moiety covalently attached to the peptide.

Techniques for the preparation of activated PEG and its conjugation to biologically active peptides are well known in the art. (E.g., see U.S. Pat. Nos. 5,643,575, 5,919,455, 5,932,462, and 5,990,237; Thompson et al., PEGylation of polypeptides, EP 0575545 B1; Petit, Site specific protein modification, U.S. Pat. Nos. 6,451,986, and 6,548,644; Harris and Chess, Effect of PEGylation on pharmaceuticals, Nature Reviews Drug Discovery 2:214-20 (2003); S. Herman et al., Poly(ethylene glycol) with reactive endgroups: 1. Modification of proteins, J. Bioactive Compatible Polymers, 10: 145-187 (1995); Y. Lu et al., Pegylated peptides III: Solid-phase synthesis with PEGylating reagents of varying molecular weight: synthesis of multiply PEGylated peptides, Reactive Polymers, 22:221-229 (1994); A. M. Felix et al., PEGylated Peptides IV: Enhanced biological activity of site-directed PEGylated GRF analogs, Int. J. Peptide Protein Res., 46:253-264 (1995); A. M. Felix, Site-specific poly(ethylene glycol)ylation of peptides, ACS Symposium Series 680(poly(ethylene glycol)): 218-238 (1997); Y. Ikeda et al., Polyethylene glycol derivatives, their modified peptides, methods for producing them and use of the modified peptides, EP 0473084 B1; G. E. Means et al., Selected techniques for the modification of protein side chains, in: Chemical modification of proteins, Holden Day, Inc., 219 (1971)).

Activated PEG, such as PEG-aldehydes or PEG-aldehyde hydrates, can be chemically synthesized by known means or obtained from commercial sources, e.g., Shearwater Polymers, (Huntsville, Ala.) or Enzon, Inc. (Piscataway, N.J.).

Useful activated PEG for purposes of the present inventive method is a PEG-aldehyde compound, such as but not limited to, a methoxy PEG-aldehyde or PEG-propionaldehyde, which is commercially available from Shearwater Polymers (Huntsville, Ala.). PEG-propionaldehyde is represented by the formula PEG-CH₂CH₂CHO. (See, e.g., U.S. Pat. No. 5,252,714). Also included within the meaning of “PEG aldehyde compound” are PEG aldehyde hydrates, e.g., PEG acetaldehyde hydrate and PEG bis aldehyde hydrate, which latter yields a bifunctionally activated structure. (See., e.g., Bentley et al., Poly(ethylene glycol) aldehyde hydrates and related polymers and applications in modifying amines, U.S. Pat. No. 5,990,237). An activated multi-branched PEG-aldehyde compound can be used (PEG derivatives comprising multiple arms to give divalent, trivalent, tetravalent, octavalent constructs). Using a 4-arm PEG derivative four pharmacologically active peptides are attached to each PEG molecule. For example, in accordance with the present invention, the peptide, e.g., a CGRP peptide, can be conjugated to a polyethylene glycol (PEG) via 1, 2, 3 or 4 amino functionalized sites of the PEG.

Heterobifunctionally activated forms of PEG are also useful as long as at least one functional PEG-aldehyde moiety is present. (See, e.g., Thompson et al., PEGylation reagents and biologically active compounds formed therewith, U.S. Pat. No. 6,552,170).

Any molecular mass for a PEG can be used as practically desired, e.g., from about 1,000 Daltons (Da) to 100,000 Da (n is 20 to 2300). The number of repeating units “n” in the PEG is approximated for the molecular mass described in Daltons. It is preferred that the combined molecular mass of PEG on an activated linker is suitable for pharmaceutical use. Thus, the combined molecular mass of the PEG molecule should not exceed about 100,000 Da.

Preferably, the combined or total molecular mass of PEG used in a PEG-conjugated peptide of the present invention is from about 3,000 Da to 60,000 Da (total n is from 70 to 1,400), more preferably from about 10,000 Da to 40,000 Da (total n is about 230 to about 910). The most preferred combined mass for PEG is from about 20,000 Da to 30,000 Da (total n is about 450 to about 680).

In accordance with the inventive method of producing a composition of matter, the inclusion in the buffer solution, in which the PEGylation reaction takes place, of an alcohol co-solvent can improve the conjugation efficiency of pharmacologically active peptides to PEG aldehyde compounds by reductive amination.

Useful aqueous buffer solutions can be made with any of the buffers known in the biochemical art that provide buffering from about pH 4 to about pH 9, with about pH 5 to about pH 7 preferred. These buffers can include, but are not limited to, acetate, citrate, 2-(N-Morpholino)-ethane sulfonic acid (MES), phosphate, N,N-Bis(2hydroxyethyl)glycine (Bicine), or borate buffers. Useful buffer concentrations may range from 5 mM to 100 mM with 10-50 mM preferred. Buffers containing a free amine group, such as TRIS, are not suitable.

In accordance with the inventive method, the buffer solution comprises an “alcohol co-solvent”, which term encompasses an alcohol solvent, in embodiments in which the (v/v) concentration of the alcohol is 100% as described below. However, in many embodiments, the concentration (v/v) of the alcohol co-solvent in an aqueous medium is about 30% to about 99%, or about 30% to about 90%, or about 30% to about 80%, or about 30% to about 70%, with about 50% to about 70% being a preferred concentration range. The skilled artisan will understand how to optimize the alcohol concentration for reductive amination reaction of a particular species of peptide with a particular species of PEG-aldehyde compound. By way of example, for some small pharmacologically active peptides (e.g., about 10-20 amino acid residues in length), an alcohol (e.g., TFE) concentration of 100% (v/v) is suitable. Alcohols that are useful as alcohol co-solvents in increasing the efficiency the reductive amination of PEG aldehyde compounds with free amine containing peptides, in accordance with the present invention, include 2,2,2-trifluoroethanol (TFE), 1,1,1,3,3,3-hexafluoro-2-propanol (HF-i-PA), and 2-trifluoromethyl-1,1,1,3,3,3-hexafluoro-2-propanol (HF-t-BuOH), although, in principle, any alcohol co-solvent based on structural Formulae IV, V and VI (below) could increase the PEGylation efficiency during reductive amination. The alcohol co-solvent comprises a structure selected from structural Formulae IV, V and VI:

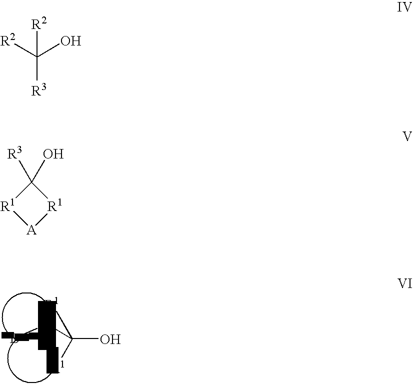

wherein R¹ is independently CFₙR⁴₂₋ₙ, n=1 to 3; wherein Formula VI comprises at least seven carbon atoms;
R⁴ is a (C₁ to C₄)linear or branched alkyl moiety, a (C₃ to C₆)cyclic alkyl moiety, or an aryl or heteroaryl moiety;
R² and R³ are independently H or R¹H;
A is (CY₂)o, wherein o equals 1 to 4, or (CY₂—X—CY₂), wherein X is O or NR⁵ and Y is independently H or F;
R⁵ is independently H or a (C₁ to C₄) linear or branched alkyl;
and wherein B is C(R⁵) or N.
(B is capable of forming a stable bicyclic structure such as bicyclo[2.2.2]octane, or the like.) In some embodiments, if X is O, Y is independently H or F; and if X is NR⁵, Y is H.

In general, the PEGylation efficiency by reductive amination of peptide sequences, such as but not limited to CGRP₁ benefits from more hydrophobic alcohols such as HF-i-PA or HP-t-BuOH. While this aspect of the present invention does not rely upon any particular mechanism of operation, it is thought that this observation relates to the increased solubility of aliphatic side chain residues in aliphatic solvents. In this context, the rank-order of aliphatic character is HP-t-BuOH>HF-i-PA>TFE. The efficiency enhancement afforded by β-fluoro alcohols is most advantageous to reductive amination reactions.

Although the method of producing a composition of matter of the present invention is not limited or dependent upon any particular mechanism of operation, there are a number of factors that may contribute to the benefits obtained in increased PEGylation efficiency. For example, these factors are hypothetically related to the strong hydrogen bonding character associated with β-fluoro alcohols such as TFE for two reasons. The first of which is the ability to dissolve polyamide structure by solvating the amide oxygen. Secondly, the strong H-bonding ability afforded by the β-fluoro alcohol should facilitate imine formation between PEG aldehyde and an amine present on the peptide in the same manner that acid does. Imine formation is a required step in the reductive amination process, and is often rate-limiting. The ability of β-fluoro alcohol to accelerate this step may be of particular advantage. In addition, polypeptide aggregation generally can bury a reactive functionality, such as a basic amine that is to undergo reductive amination with PEG aldehyde. Consequently, another rationale that may explain the increased efficiency for PEGylation being described herein is that the alcohol co-solvent (e.g., TFE) decreases oligomerization that would otherwise limit the amount of available basic amine that is required to react with PEG aldehyde. In the context of the current invention, the change of conformation for these terminal 3 amino acids could increase the amount of free reactive amine that would again lead to increased PEGylation efficiency.

The invention will be described in greater detail by reference to the following examples. These examples are not to be construed in any way as limiting the scope of the present invention.

### EXAMPLES

### Example 1

### Peptide Synthesis

A series of CGRP peptide analogs were synthesized by conventional solid phase peptide synthetic techniques for the purpose of introducing site-selective conjugation sites for activated PEG.

CGRP peptide synthesis. Briefly, the following protocol was used to generate CGRP analogs. N^{α}-Fmoc, side-chain protected amino acids and Rink amide resin were used. Representative side-chain protection strategies were: Asp(OtBu), Arg(Pbf), Cys(Acm), Glu(OtBu), Glu(O2-PhiPr), His(Trt), Lys(N^{ε}-Boc), Lys (N^{α}-Mtt), Ser(OtBu), Thr(OtBu), Trp(Boc) and Tyr(OtBu). CGRP peptide derivatives were synthesized in a stepwise manner on an AB1433 peptide synthesizer by SPPS using O-Benzotriazole-N,N,N′,N′-tetramethyl-uronium-hexafluoro-phosphate (HBTU)/N,N-diisopropylethylamine (DIEA)/N,N-dimethylformamide (DMF) coupling chemistry at 0.2 mmol equivalent resin scale (Fmoc-deprotected Rink amide resin). For each coupling cycle, 1 mmol N^{α}-Fmoc-amino acid, 4 mmol DIEA and 1 mmol equivalents of HBTU were used. The concentration of the HBTU-activated Fmoc amino acids was 0.5 M in DMF, and the coupling time was 45 min. Fmoc deprotections were carried out with two treatments using a 30% piperidine in DMF solution first for 2 min and then for an additional 20 min.

Lactam Formation. In some of the CGRP peptides, side-chain to side-chain lactam formation was carried out on the assembled N-terminally Fmoc-protected peptide resin. The peptide-resin was solvated in DCM for 30 mins, and drained. The Mtt and 2-PhiPr groups (protecting group at the specified lactam bond forming sites) were removed with 1% TFA in DCM solution containing 5% TIS. Treatment of the peptide-resin with the 1% TFA in DCM solution was repeated 8 times in 30 min increments, and each treatment was followed by extensive DCM washes. The liberate carboxyl and amino groups were then condensed by the addition of 5 equiv of 0.5M benzotriazole-1-yl-oxy-tris-pyrrolidino-phosphonium hexafluorophosphate (PyBOP) and 10 equiv of DIEA in DMF were added to the peptide resin, and left for 24 h. The resin was then wash thoroughly with DMF, DCM, and DCM/MeOH, and dried.

Side Chain Deprotection and Cleavage from Resin. Following synthesis and modification, the resin was then drained, and washed with DCM, DMF, DCM, and then dried in vacuo. The peptide-resin was deprotected and released from the resin by treatment with a trifluoroacetic acid (TFA)/1,2-ethanedithiol (EDT)/triisopropyl-silane (TIS)/H₂O (92.5:2.5:2.5:2.5 v/v) solution at room temperature for 90 min. The volatiles were then removed with a stream of nitrogen gas, the crude peptide precipitated twice with diethyl ether and collected by centrifugation.
Reversed-Phase HPLC Purification. Reversed-phase high-performance liquid chromatography was performed on an analytical (C18, 5 μm, 0.46 cm×25 cm) or a preparative (C18, 10 μm, 2.2 cm×25 cm) column. Chromatographic separations were achieved using linear gradients of buffer B in A (A=0.1% aqueous TFA; B=90% aq. ACN containing 0.09% TFA) typically 5-95% over 35 min at a flow rate of 1 mL/min for analytical analysis and 5-65% over 90 min at 20 mL/min for preparative separations. Analytical and preparative HPLC fractions were characterized by ESMS and photodiode array (PDA) HPLC, and selected fractions combined and lyophilized.

CGRP₁ binding experiments. CGRP₁ binding assays were set up in 96-well plates at room temperature containing: 110 μl binding buffer (20 mM Tris-HCl, pH7.5, 5.0 mM MgSO₄, 0.2% BSA [Sigma], 1 tablet of Complete™/50 ml buffer [protease inhibitor]); 20 μl test compound (10×); 20 μl ¹²⁵I-hαCGRP (Amersham Biosciences) (10×); and 50 μl human neuroblastoma cell (SK-N-MC) membrane suspension (10 μg per well, PerkinElmer). The plates were incubated at room temperature for 2 hour with shaking at 60 rpm, then the contents of each well were filtered over 0.5% polyethyleneimine (PEI)-treated (at least one hour) GF/C 96-well filter plates. The GF/C filter plates were washed 6 times with ice-cold 50 mM Tris, pH 7.5 and dried in an oven at 55 C for 1 hour. The bottoms of the GF/C plates were then sealed, and 40 μl Microscint 20 was added to each well, then the tops of the GF/C plates were sealed with TopSeal™-A, a press-on adhesive sealing film, and the GF/C plates were counted with TopCount NXT (Packard). The data were analyzed by using ActivityBase (IDBS) or Prizm (GraphPad) software. Results of CGRP₁ binding assays are not shown, but are found in U.S. Non-provisional patent application Ser. No. 11/584,177, titled “CGRP peptide antagonists and conjugates” by Gegg et al., filed Oct. 19, 2006, which claims the benefit of priority of U.S. Provisional Patent Application No. 60/729,083, filed Oct. 21, 2005, both of which are incorporated herein by reference in their entireties.

CGRP peptide antagonist activity. Un-conjugated CGRP peptide analogs were screened in an in vitro CGRP₁ receptor mediated cAMP assay to determine intrinsic potency prior to PEGylation. The results of such in vitro cAMP-based assays are not shown, comparing native human αCGRP (8-37) peptide antagonist VTHRLAGLLSRSGGVVKNNFVPTNVGSKAF-NH₂ (SEQ ID NO: 1) with a variety of other analogs designed to enhance potency at the CGRP₁ receptor, permit site-selective conjugation and/or facilitate handling in vitro, but these results are found in U.S. Non-provisional patent application Ser. No. 11/584,177, titled “CGRP peptide antagonists and conjugates” by Gegg et al., filed Oct. 19, 2006, which claims the benefit of priority of U.S. Provisional Patent Application No. 60/729,083, filed Oct. 21, 2005, both of which are incorporated herein by reference in their entireties.

Briefly, the in vitro cAMP assay employed a human neuroblastoma-derived cell line (SK-N-MC). SK-N-MC cells express CRLR and RAMP1, which form CGRP₁ receptor. CGRP₁ receptor is a G-protein coupled receptor linked to the stimulatory G-protein, Gₛ. Upon CGRP binding to the receptor, cAMP levels increase, mediated by Gₛ activation of adenylyl cyclase and cAMP-dependent protein kinase (PKA). An ELISA assay was developed to measure the level of cAMP accumulated in treated SK-N-MC cells as a screening system for the identification of CGRP antagonists. Briefly, two days before the assay, the cells were plated in 96-well plates in growth medium. On the day of the assay cells were rinsed with PBS followed by the addition of 60 μl of plasma free medium for 15 minutes at room temperature. CGRP peptide antagonists were then added at varying concentrations in 20 μl buffer followed by a 5 minute incubation at 37° C. The CGRP agonist, αCGRP, was then added in 20 μl buffer to each well followed by a 5 minute incubation at 37° C. The medium was then removed and the cells were lysed with 100 μl lysis buffer for 30 minutes at 37° C. for 30 minutes. Using a Tropix ELISA kit (Applied Biosystems, Foster City, Calif.), a cAMP-AP conjugate was diluted (1:100) and added (30 μl) to each well of a 96-well ELISA plate. An aliquot of the lysis buffer from the cell plate was then added to the ELISA plate followed by an aliquot of the cAMP antibody. This mixture was incubated for 1 hour at room temperature with gentle shaking. The ELISA plate was then washed 6× with wash buffer followed by the additions of a substrate enhancer. This mixture was incubated for 30 minutes at room temperature and then read on a MicroBeta Jet or EnVision plate reader. The results were analyzed using Prism (GraphPad).

### Example 2

Antagonist activity by vehicle-conjugated CGRP peptide antagonists. A variety of CGRP analogs were engineered for vehicle conjugation through several different conjugating chemistries. Monofunctional 20 kDa methoxy PEG derivatives were conjugated to the appropriate CGRP peptide analogs resulting in amine linkages. The specific conjugation site was also varied to include the CGRP peptide N-terminus or amino acid positions 23, 24 or 25 (relative to the native human αCGRP sequence) in the CGRP peptide hinge region.

Results of in vitro CGRP₁ receptor-mediated cAMP assays (see, Example 1) comparing a variety of PEG-conjugated CGRP peptide antagonists are not shown. However, these results are found in U.S. Non-provisional patent application Ser. No. 11/584,177, titled “CGRP peptide antagonists and conjugates” by Gegg et al., filed Oct. 19, 2006, which claims the benefit of priority of U.S. Provisional Patent Application No. 60/729,083, filed Oct. 21, 2005, both of which are incorporated herein by reference in their entireties.

Briefly, the amine-linked PEG-peptides were derived from CGRP peptide antagonists, typically with their N-termini acylated and a single primary amine engineered into the designated conjugation site.

The activated PEG derivative used was a 20 kDa methoxy PEG-aldehyde (in PEG-ald) and conjugation was achieved by reductive amination in the presence of sodium cyanoborohydride. The CGRP peptide was dissolved at 2 mg/ml in an amine-free buffer (20 mM sodium phosphate, pH 6), the mPEG-ald was added in stoichiometric excess (about 5-fold) and sodium cyanoborohydride was added to a final concentration of 10 mM. The reaction mixture was stirred at room temperature for 24 to 48 hours and was monitored by reverse phase high-pressure liquid chromatography (RP-HPLC). Upon completion, the reaction was quenched by a 4- to 5-fold dilution into 20 mM sodium acetate buffer, pH 4. Purification was achieved by preparative cation-exchange chromatography, eluting with a linear 0-500 mM sodium chloride gradient. The eluted PEG-peptide was identified by RP-HPLC and sodium dodecyl sulfate polyacrylamide gel electrophoresis (SDS-PAGE), and was concentrated and dialyzed into 10 mM sodium acetate, 5% sorbitol, at pH 4. Purities of greater than 99% were determined for all the final pools by RP-HPLC. Peptide mapping and sequencing were used to confirm conjugation of PEG at each of the intended conjugation sites.

The invention is not limited to particular peptide sequence modifications or PEG molecules. For example, efficacious PEG-peptide conjugates can be achieved at other positions in the hinge region or in either of the CGRP₁ receptor binding regions. Further, the introduction of reactive conjugation sites does not rely solely on sequence substitutions with residues bearing secondary amine moieties, but can include insertions between existing residues, or addition of different reactive amino acids. Moreover, since this invention can utilize synthetic peptides, numerous chemical strategies can be employed to achieve PEG conjugation at preferred sites in the peptide of interest.

### Example 3

Co-solvent mediated conjugation of vehicle to CGRP peptide antagonists. Some CGRP peptides developed to resist proteolysis proved to be considerably less soluble in aqueous buffer medium than their parent peptides (e.g., relatively soluble parent WVTHRLAGLLSRSGGVVRKNFVPTDVGPFAF-NH₂ (SEQ. ID NO:7), in the aqueous conjugation buffers used for coupling vehicle to the peptide (see, Example 2 herein). In an attempt to better solubilize these less soluble peptides during the conjugation reaction, and thereby improve conjugate yields, a variety of alcohol co-solvents were tested in the buffer solution for the conjugation reaction.

Briefly, a series of reductive amination conjugation reactions using 20 kDa mPEG-ald and the relatively soluble CGRP peptide WVTHRLAGLLSRSGGVVRKNFVPTDVGPFAF-NH₂ (SEQ. ID NO:7) were prepared as described in Example 2, with the exception that the alcohols: methanol (MeOH), ethanol (EtOH), isopropanol (IPA) and tri-fluoroethanol (TFE) were each added at either 25% or 50% (v/v) ratio. The progress of each reaction was monitored by RP-HPLC after 20 hrs. Site-specific PEG conjugation is indicated below at the underlined residue, except, as in Table 3, where it is indicated by a parenthetic immediately following the residue.

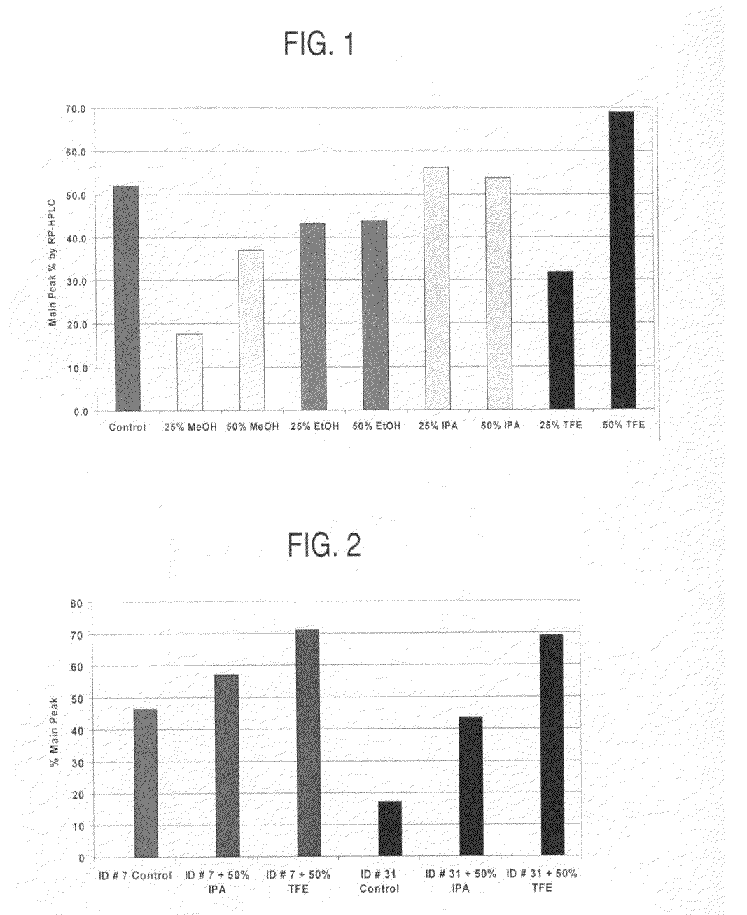

The mono-PEGylated peptide product Ac-WVTHRLAGLLSRSGGVVRKNFVPTDVGPFAF-NH₂ (SEQ ID NO: 15) was quantitated by integration of the RP-HPLC chromatograms and reported as % Product Peak (FIG. 1). These results demonstrate an inhibitory effect by MeOH and EtOH on PEGylation of this relatively soluble CGRP peptide whereas IPA showed little effect and surprisingly TFE showed both positive and negative effects, depending on concentration.

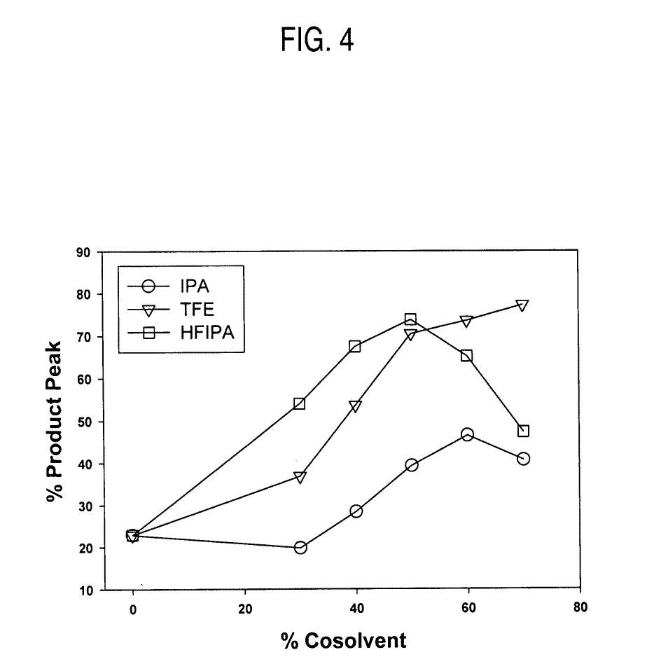

Next, a comparison was made between the relatively soluble CGRP peptide Ac-WVTHRLAGLLSRSGGVVRKNFVPTDVGPFAF-NH₂ (SEQ. ID NO:7) and a relatively insoluble CGRP peptide Ac-WVTH(Cit)LAGLLS(Cit)SGGVVRKNFVPTDVGPFAF-NH₂ (SEQ ID NO:31). In this experiment both peptides were PEGylated by reductive alkylation, as described in Example 2, using 20 kDa mPEG-ald in the presence of either 50% IPA or 50% TFE. The progress of each reaction was monitored by RP-HPLC after 20 hrs. The respective mono-PEGylated peptide product Ac-WVTHRLAGLLSRSGGVVRKNFVPTDVGPFAF-NH₂ (SEQ ID NO: 15) or Ac-WVTH(Cit)LAGLLS(Cit)SGGVVRKNFVPTDVGPFAF-NH₂ (SEQ ID NO:32) was quantitated by integration of the RP-HPLC chromatograms and reported as % Product Peak (FIG. 2). These results duplicate the modest improvement in product SEQ ID NO:15 yield for the more soluble CGRP peptide also observed in FIG. 4. Surprisingly, both co-solvents provided a much more dramatic improvement in PEGylation efficiency for the relatively insoluble CGRP peptide SEQ ID NO:31, showing increases in product yields of 2.6-fold with IPA and 4.1-fold with TFE.

Over 42 relatively insoluble CGRP peptides have been PEGylated by this method in the presence of 50% TFE with good product yield. At least 34 of these conjugates were produced in excess of 60% product yield (Table 3).

In order to determine the optimal concentrations of co-solvent for PEGylation of different CGRP peptides titration experiments were performed from 30-70% co-solvent using either IPA, TFE or hexafluoro-isopropyl alcohol (HFIPA or HF-i-PA). The PEGylation reactions were prepared as described in Example 2 for reductive alkylation with the three alcohols added at their stated concentrations. The progress of each reaction was monitored by RP-HPLC after 20 hrs. The mono-PEGylated peptide product was quantitated by integration of the RP-HPLC chromatograms and reported as % Product Peak. Unfortunately, HFIPA proved partially immiscible in the aqueous reaction buffer rendering the PEGylation results HFIPA cosolvent difficult to use in the existing process.

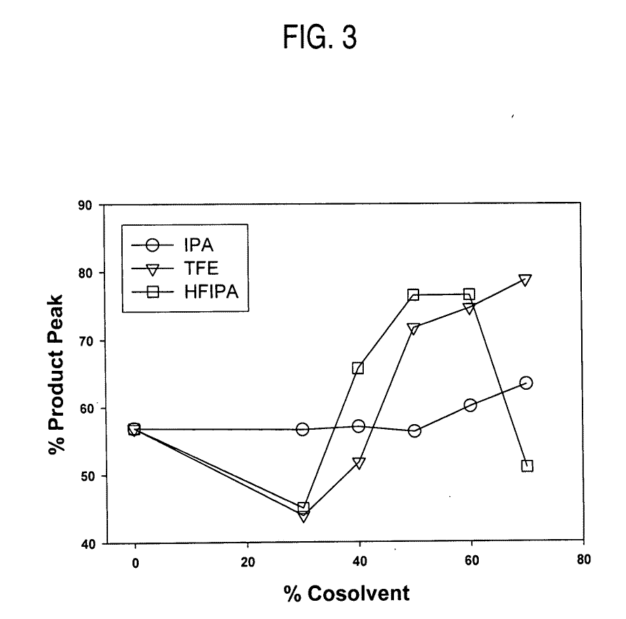

FIG. 3 shows the effects of IPA, TFE, and HFIPA on the relatively soluble CGRP peptide SEQ ID NO:7 examined previously. These data show little effect from IPA until the concentration exceeds 50%. At 70% IPA, a modest 1.1-fold increase in product yield of SEQ ID NO:15 was observed. However, 70% TFE showed a more significant increase in product yield of 1.4-fold. HFIPA showed an apparent maximum effect between about 50-60%.

FIG. 4 shows the effect of IPA, TFE, and HFIPA on a relatively insoluble CGRP peptide Ac-WVTH[Cit]LAGLLS[Cit]SGGVV[hArg]KNFVPTDVGPFAF-NH₂ (SEQ ID NO:2). These data show a much more pronounced effect for all three co-solvents. Where IPA produced a maximal effect at about 60% IPA of 1.8-fold increase in product yield for PEGylated CGRP peptide Ac-WVTH[Cit]LAGLLS[Cit]SGGVV[hArg]K^{(20kDa MeO-PEG)}NFVPTDVGPFAF-amide (SEQ ID NO:5). With TFE, the results were even more dramatic, showing a continued increase in product yield peaking at about 70% TFE and 3.4-fold. HFIPA also induced a significant increase in product yield (˜3.2-fold) at about 50% cosolvent, but yields rapidly dropped off at higher HFIPA concentrations.

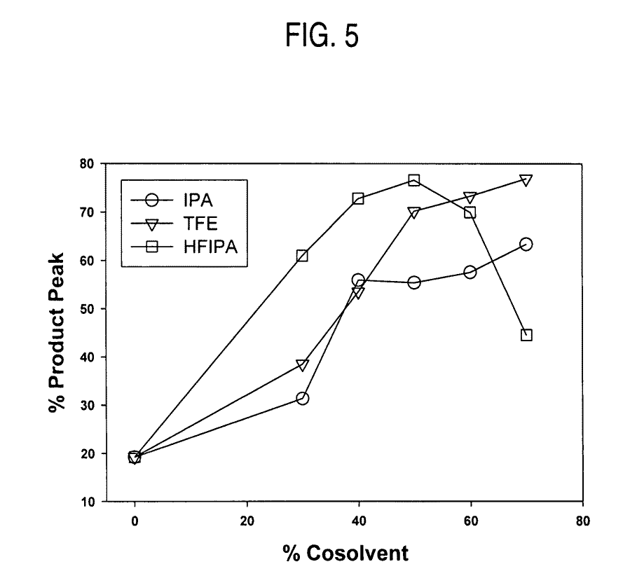

FIG. 5 shows the effect of IPA, TFE, and HFIPA on another relatively insoluble CGRP peptide Ac-WVTH[Cit]LAGLLS[Cit]SGGVV[hArg]KNFVPTDVG[Oic]FAF-NH₂ (SEQ ID NO:3). These data show a much more pronounced effect for all three co-solvents. Where IPA produces a maximal effect at about 70% IPA of 3.3-fold increase in product yield of PEGylated CGRP peptide Ac-WVTH[Cit]LAGLLS[Cit]SGGVV[hArg]K^{(20 kDa MeO-PEG)}NFVPTDVG[Oic]FAF-amide (SEQ ID NO:6). With TFE, the maximum yield was achieved at about 70% TFE with 4.0-fold increase. Again, the HFIPA gives a maximal effect of ˜4.0-fold increased yield at 50% cosolvent, but then yields quickly drop off at higher HFIPA concentrations.

Table 3 shows representative yields for a variety of PEGylated CGRP peptides PEGylated in the presence of 50% TFE as described herein above. In almost all cases, the reaction yields were between about 50% to about 70%, a dramatic increase from the typical yield of less than 20% seen with the PEGylation of, e.g., SEQ ID NO:31, in the absence of the alcohol co-solvent (See, FIG. 2).

TABLE 3
Representative yields for a variety of PEGylated CGRP peptides
PEGylated in the presence of 50% TFE as described herein above.
Underlined boldface amino acid residues, if any, indicate
cyclization between the first underlined boldface residue
and the second underlined boldface residue in a sequence.

| | | Main |
|---|---|---|
| SEQ | | Peak |
| ID NO: | Peptide Sequence | % |
| 32 | Ac-WVTH[Cit]LAGLLS[Cit]SGGVVRK^{(20kDa MeO-PEG)} | 68 |
| | NFVPTDVGPFAF-amide | |
| 8 | Ac-WVTHRLAGLASRSGGVVRK^{(20kDa MeO-PEG)}NFVPT | 68 |
| | DVGPFAF-amide | |
| 9 | Ac-WVTHRLAGLAS[Cit]SGGVVRK^{(20kDa MeO-PEG)}NFVPT | 71 |
| | DVGPFAF-amide | |
| 10 | Ac-WVTH[Cit]LAGLAS[Cit]SGGVVRK^{(20kDa MeO-PEG)} | 69 |
| | NFVPTDVGPFAF-amide | |
| 11 | Ac-WVTHRLAGLLSRPGGVVRK^{(20kDa MeO-PEG)} | 71 |
| | NFVPTDVGPFAF-amide | |
| 12 | Ac-WVTHRLAGLASRPGGVVRK^{(20kDa MeO-PEG)} | 80 |
| | NFVPTDVGPFAF-amide | |
| 13 | Ac-WVTH[Cit]LAGLASRPGGVVRK^{(20kDa MeO-PEG)} | 61 |
| | NFVPTDVGPFAF-amide | |
| 14 | Ac-WVTH[Cit]LAGLLSRPGGVVRK^{(20kDa MeO-PEG)} | 64 |
| | NFVPTDVGPFAFd | |
| 16 | Ac-WVTH[Cit]LAGLLPRSGGVVRK^{(20kDa MeO-PEG)} | 67 |
| | NFVPTDVGPFAF-amide | |
| 17 | Ac-WVTHRLAGLLPRSGGVVRK^{(20kDa MeO-PEG)} | 55 |
| | NFVPTDVGPFAF-amide | |
| 18 | Ac-WVTHQLAGLLSQSGGVV[hArg]K^{(20kDa MeO-PEG)} | 72 |
| | NFVPTDVGPFAF-amide | |
| 5 | Ac-WVTH[Cit]LAGLLS[Cit]SGGVV[hArg]K^{(20kDa MeO-PEG)} | 63 |
| | NFVPTDVGPFAF-amide | |
| 19 | Ac-WVTHRLAGLLSRSGGVVR[4Apa]^{(20kDa MeO-PEG)} | 78 |
| | NFVPTDVGPFAF-amide | |
| 20 | Ac-WVTHRLAGLLS[Cit]SGG VVRK^{(20kDa MeO-PEG)} | 69 |
| | NFVPTDVGPFAF-amide | |
| 15 | Ac-WVTHRLAGLLSRSGGVVRK^{(20kDa MeO-PEG)} | 67 |
| | NFVPTDVGPFAF-amide | |
| 21 | Ac-WVTHR[NMeLeu]AGLLSR[NMeSer]GGVVRK^{(20kDa MeO-PEG)} | 66 |
| | NFVPTDVGPFAF-amide | |
| 22 | Ac-WVEHRLKGLLSRSGGVVRK^{(20kDa MeO-PEG)} | 65 |
| | NFVPTDVGPFAF-amide | |
| 23 | Ac-WVTHRLEGLLKRSGGVVRK^{(20kDa MeO-PEG)} | 60 |
| | N FVPTDVGPFAF-amide | |
| 24 | Ac-[1-Nal]VTH[Cit]LAGLLS[Cit]SGGVVRK^{(20kDa MeO-PEG)} | 14 |
| | NFVPTDVGPFAF-amide | |
| 25 | Ac-WVEH[hArg]LKGLLS[Cit]SGGVVRK^{(20kDa MeO-PEG)} | 59 |
| | NFVPTDVGPFAF-amide | |
| 26 | Ac-[1-Nal]VEH[hArg]LKGLLS[Cit]SGGVVRK^{(20kDa MeO-PEG)} | 66 |
| | NFVPTDVGPFAF-amide | |
| 27 | Ac-[Aib]WVEH[hArg]LKGLLS[Cit]SGGVVRK^{(20kDa MeO-PEG)} | 70 |
| | NFVPTDVGPFAF-amide | |
| 28 | Ac-WVTH[hCit]LAGLLS[hCit]SGGVV[hArg]K^{(20kDa MeO-PEG)} | 63 |
| | NFVPTDVGPFAF-amide | |
| 29 | Ac-WVTH[hArg]LAGLLS[hCit]SGGVV[hArg]K^{(20kDa MeO-PEG)} | 61 |
| | NFVPTDVGPFAF-amide | |
| 30 | Ac-WVTH[hArg]LAGLLS[Cit]SGGVV[hArg]K^{(20kDa MeO-PEG)} | 60 |
| | NFVPTDVGPFAF-amide | |
| 33 | Ac-WVTH[hArg]LAGLLS[hArg]SGGVV[hArg]K^{(20kDa MeO-PEG)} | 56 |
| | NFVPTDVGPFAF-amide | |
| 34 | Ac-WVTHQLAGLLS[Cit]SGGVVR[hArg]K^{(20kDa MeO-PEG)} | 64 |
| | )NFVPTDVGPFAF-amide | |
| 35 | Ac-WVTH[Cit]LAGLLSQSGGVVR[hArg]K^{(20kDa MeO-PEG)} | 63 |
| | NFVPTDVGPFAF-amide | |
| 36 | Ac-WVTH[Cit]LAGLLS[hArg]SGGVVR[hArg]K^{(20kDa MeO-PEG)} | 61 |
| | NFVPTDVGPFAF-amide | |
| 37 | Ac-WVTH[hCit]LAGLLS[hArg]SGGVVR[hArg]K^{(20kDa MeO-PEG)} | 63 |
| | NFVPTDVGPFAF-amide | |
| 38 | Ac-WVEHRLKGLLS[Cit]SGGVVR[hArg]K^{(20kDa MeO-PEG)} | 66 |
| | NFVPTDVGPFAF-amide | |
| 39 | Ac-WVEH[Cit]LKGLLS[Cit]SGGVVR[hArg]K^{(20kDa MeO-PEG)} | 65 |
| | NFVPTDVGPFAF-amide | |
| 40 | Ac-WVEH[hArg]LKGLLS[Cit]SGGVV[hArg]K^{(20kDa MeO-PEG)} | 63 |
| | NFVPTDVGPFAF-amide | |
| 41 | Ac-WVEHRLKGLLS[hArg]SGGVVR[hArg]K^{(20kDa MeO-PEG)} | 48 |
| | NFVPTDVGPFAF-amide | |
| 42 | Ac-WVEHRLKGLLSQSGGVVR[hArg]K^{(20kDa MeO-PEG)} | 61 |
| | NFVPTDVGPFAF-amide | |
| 6 | Ac-WVTH[Cit]LAGLLS[Cit]SGGVV[hArg]K^{(20kDa MeO-PEG)} | 58 |
| | NFVPTDVG[Oic]FAF-amide | |
| 44 | Ac-WVTH[Cit]LAGLLS[Cit]SGGVV[hArg]K^{(20kDa MeO-PEG)} | 66 |
| | NFVPTDVGP[1-Nal]AF-amide | |
| 45 | Ac-WVTH[Cit]LAGLLS[Cit]SGGVV[Guf]K^{(20kDa MeO-PEG)} | 58 |
| | NFVPTDVGPFAF-amide | |
| 46 | Ac-WVTH[Cit]LAGLLS[Cit]SGGVV[hArg]K^{(20kDa MeO-PEG)} | 66 |
| | NFVPTDVGP[Bip]AF-amide | |
| 47 | Ac-WVTH[hArg]LAGLLS[Cit]SGGVV[hArg]K^{(20kDa MeO-PEG)} | 70 |
| | NFVPTDVGP[2-Nal]AF-amide | |
| 48 | Ac-WVTH[Cit]LAGLLS[Cit]SGGVV[hArg]K^{(20kDa MeO-PEG)} | 61 |
| | NFVPTDVGP[Igl]AF-amide | |
| 49 | Ac-WVTH[Cit]LAGLLS[Cit]SGGVV[hArg]K^{(20kDa MeO-PEG)}N[pl- | 59 |
| | Phe]VPTDVGP[pI-Phe]AF-amide | |

Together, these data illustrate a significant and unexpected efficiency benefit to be realized by utilizing co-solvents in reductive amination PEGylation reactions for pharmacologically active peptides.

The foregoing being illustrative but not an exhaustive description of the embodiments of the present invention, the following claims are presented.

We claim:

1. A method of producing a composition of matter, comprising the steps of:
(a) obtaining a pharmacologically active peptide; and
(b) conjugating the obtained pharmacologically active peptide to a pharmaceutically acceptable polyethylene glycol (PEG) by reacting the peptide with a PEG-aldehyde compound at a free amine moiety on the peptide in a buffer solution comprising an alcohol co-solvent, whereby the composition of matter is produced.

2. The method of claim 1, wherein the alcohol co-solvent comprises a structure selected from structural Formulae IV, V and VI: 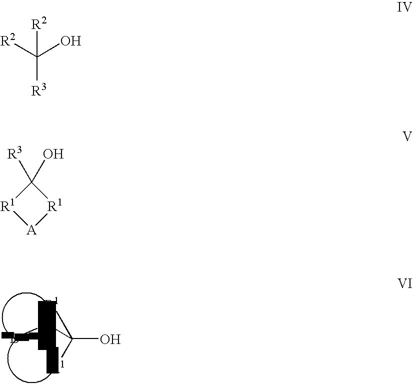
wherein R¹ is independently CFₙR⁴₂₋ₙ, n=1 to 3; wherein Formula VI comprises at least seven carbon atoms;
R⁴ is a (C₁ to C₄)linear or branched alkyl moiety, a (C₃ to C₆)cyclic alkyl moiety, or an aryl or heteroaryl moiety;
R² and R³ are independently H or R¹H;
A is (CY₂)o, wherein o equals 1 to 4, or (CY₂—X—CY₂), wherein X is O or NR⁵ and Y is independently H or F;
R⁵ is independently H or a (C₁ to C₄) linear or branched alkyl;
and wherein B is C(R⁵) or N.

3. The method of claim 2, wherein if X is O, Y is independently H or F; and if X is NR⁵, Y is H.

4. The method of claim 1, wherein the alcohol co-solvent is selected from 2,2,2-trifluoroethanol (TFE), 1,1,1,3,3,3-hexafluoro-2-propanol (HF-i-PA), and 2-trifluoromethyl-1,1,1,3,3,3-hexafluoro-2-propanol (HF-t-BuOH).

5. The method of claim 1, wherein the concentration of the alcohol co-solvent in the buffer solution is about 30% to about 100% (v/v).

6. The method of claim 1, wherein the pharmacologically active peptide is selected from insulin, interleukin (IL)-1ra, leptin, soluble tumor necrosis factor receptor type 1, soluble tumor necrosis factor receptor type 2, keratinocyte growth factor, erythropoietin, thrombopoietin (TPO), TPO-mimetic peptides, granulocyte colony-stimulating factor, darbepoietin, glial cell line-derived neurotrophic factor, calcitonin, amylin, adrenomedullin, calcitonin gene-related peptide (CGRP) peptide antagonists, BAFF antagonist peptides, ang-2-binding peptides, NGF-binding peptides, myostatin-binding peptides, and toxin peptides.
